(19) United States
(12) Patent Application Publication
(10) Pub. No.: US 2024/0207612 A1
(43) Pub. Date: Jun. 27, 2024

(54) DIAGNOSIS SYSTEM FOR SARCOPENIA AND FUNCTIONAL ELECTRICAL STIMULATION THERAPY SYSTEM USING ELECTROMYOGRAPHY SIGNAL

(71) Applicant: EXOSYSTEMS INC., Seongnam-si (KR)

(72) Inventors: Hooman LEE, Seoul (KR); Kwangsub SONG, Gwangju-si (KR); Sang Ui CHOI, Seoul (KR)

(21) Appl. No.: 17/915,456

(22) PCT Filed: May 9, 2022
(86) PCT No.: PCT/KR2022/006531

§ 371 Date: Sep. 28, 2022

(30) Foreign Application Priority Data
May 7, 2021 (KR) 10-2021-0059401
Mar. 11, 2022 (KR) 10-2022-0030403
Mar. 11, 2022 (KR) 10-2022-0030408

(51) Int. Cl. A61N 1/36

(52) U.S. Cl. CPC A61N 1/36031; A61N 1/36003

(57) ABSTRACT

A sarcopenia diagnostic system of present invention comprises, an electrical stimulation and measurement unit configured to apply multi-frequency electrical stimulation to the body and measure a multi-frequency impulse response signal m-FIRS to the multi-frequency electrical stimulation, a response signal analysis unit configured to remove noise and distortion from the multi-frequency impulse response signal m-FIRS to obtain an involuntary muscle contraction signal, and configured to extract a feature vector in each of time domain and frequency domain from the involuntary muscle contraction signal, and an artificial intelligence model learning unit receiving the extracted feature vector as input, and generates a classification for muscle strength and muscular endurance from the feature vector through artificial intelligence-based model learning to diagnose sarcopenia, wherein the multi-frequency impact response signal m-FIRS is provided in units of a plurality of segments divided by frequency.

### CROSS-REFERENCE TO RELATED APPLICATIONS

This application claims the benefit under 35 U.S.C. section 371, of PCT International Application No. PCT/KR2022/006531, filed on May 9, 2022, which claims foreign priority to Korean Patent Application No. 10-2021-0059401, filed on May 7, 2021, Korean Patent Application No. 10-2022-0030408, filed on Mar. 11, 2022, and Korean Patent Application No. 10-2022-0030403, filed on Mar. 11, 2022, in the Korean Intellectual Property Office, the disclosures of which are hereby incorporated by reference in their entireties.

This work was supported by the Korea Medical Device Development Fund grant funded by the Korea government (the Ministry of Science and ICT, the Ministry of Trade, Industry and Energy, the Ministry of Health Welfare, the Ministry of Food and Drug Safety) (Project Number: 1711138172).

### BACKGROUND

Embodiments of the present disclosure described herein relate to a sarcopenia diagnosis system and treatment system, and more particularly, to a sarcopenia diagnosis system using a multi-frequency electrical stimulation-based response signal and an artificial intelligence learning model, and an electrical stimulation treatment system that generates functional electrical stimulation FES signals based on electromyography signals.

Sarcopenia refers to a disease in which muscle mass, strength, and muscle function all decrease. The causes of sarcopenia vary from person to person, but the most common causes are low protein intake, lack of exercise, and poor exercise method. In particular, the rate of sarcopenia is very high due to insufficient intake and absorption of essential amino acids. Another common cause of sarcopenia is the hormone deficiency associated with aging.

In addition to diseases occurring in the muscles itself, sarcopenia is often secondary to degenerative diseases such as diabetes, infectious diseases, acute and chronic diseases such as cancer, and spinal stenosis. It is known that sarcopenia occurs with a high frequency when chronic diseases of the heart, lungs, kidneys, or hormonal diseases occur.

Symptoms of sarcopenia include muscle weakness, weakness in the lower extremities, and fatigue. Muscle quality naturally decreases with age, but in sarcopenia, muscle quality (hereinafter, MQ) is excessively reduced even when age or gender is taken into account, resulting in decreased physical function and increased health risks and mortality.

Muscle weakness often occurs before sarcopenia. If muscle weakness or sarcopenia occurs, it is most important to find the factors affecting the worsening of the symptoms, identify the accompanying diseases, and then eliminate the causes. Patients with sarcopenia have a slow gait, low muscular endurance, difficulty in daily living, and frequent need for help from others. In addition, osteoporosis, falls, and fractures easily occur. As the muscle's blood and hormone buffering function is reduced, the basal metabolic rate is reduced, chronic diseases are difficult to control, and diabetes and cardiovascular diseases can be easily exacerbated.

In order to diagnose a disease such as sarcopenia, it is important to accurately diagnose the condition of the muscle. However, at present, the muscle condition is mainly measured by experts using expensive equipment. Therefore, a technique for accurately measuring the muscle state is required even by a household or a non-specialist.

Electromyography EMG, which measures the degree of muscle activity by measuring the potential difference generated in muscle cells when the muscle is activated, is widely used not only in the medical field but also in the biomechanics field. EMG technology has been developed according to the configuration of an electrode that measures the potential difference of an activated muscle, and the commonly used form is an electrode-type EMG device attached to the skin surface. In addition, electrical stimulation technology is a technology that artificially induces muscle contraction by applying electrical stimulation in the form of a constant current or constant voltage to the muscle. Electrical stimulation technology has been mainly developed into functional electrical stimulation FES technology that supplements and replaces weakened or lost muscle functions.

Functional electrical stimulation FES has been generally known as the most effective rehabilitation treatment available in hospitals. For treatment using functional electrical stimulation FES, rehabilitation specialists apply electrical stimulation to the affected area while voluntary muscle contraction occurs. Rehabilitation specialists visually determine whether the patient is maintaining or starting muscle contraction, and then turning on the power of the FES device. In general FES equipment, when the user applies more than a certain amount of force, it is driven in such a way that electrical stimulation is emitted.

Therefore, for rehabilitation using FES, one rehabilitation specialist has no choice but to handle one patient. Therefore, when there are a large number of patients, a shortage of manpower is inevitable even if the FES equipment is used. Even in the rehabilitation treatment in a home environment using the FES device, it is difficult to proceed with the most effective FES rehabilitation treatment without a rehabilitation specialist. Rehabilitation specialists have to Fig. out whether the patient is maintaining or starting voluntary muscle contraction, so it is difficult to treat multiple people. In the conventional product or technology, when the user applies more than a certain amount of force, since the electrical stimulation is controlled to come out, the equipment does not recognize whether the muscle is contracted after the electrical stimulation is output. When a patient's voluntary muscle contraction occurs, a device for automatically applying electrical stimulation is required, so a technique for analyzing and judging an input signal is required. Accordingly, there is a constant need for an FES technology that enables an effective FES rehabilitation treatment for a patient without a rehabilitation specialist.

### SUMMARY

Embodiments of the present disclosure is to solve the above-described technical problem, and the present invention is to provide an electrical stimulation treatment system that generates functional electrical stimulation FES based on a muscle stimulation signal. When a muscle is stimulated by electrical stimulation, involuntary muscle contraction also occurs. An object of the present invention is to provide an effective FES treatment system using a voluntary muscle contraction signal based on a preprocessing technique for distinguishing between involuntary muscle contraction and voluntary muscle contraction.

According to an embodiment of a sarcopenia diagnostic system of present invention comprises, an electrical stimulation and measurement unit configured to apply multi-frequency electrical stimulation to the body and measure a multi-frequency impulse response signal m-FIRS to the multi-frequency electrical stimulation, a response signal analysis unit configured to remove noise and distortion from the multi-frequency impulse response signal m-FIRS to obtain an involuntary muscle contraction signal, and configured to extract a feature vector in each of time domain and frequency domain from the involuntary muscle contraction signal, and an artificial intelligence model learning unit receiving the extracted feature vector as input, and generates a classification for muscle strength and muscular endurance from the feature vector through artificial intelligence-based model learning to diagnose sarcopenia, wherein the multi-frequency impact response signal m-FIRS is provided in units of a plurality of segments divided by frequency.

In this embodiment, the response signal analysis unit includes, an electrical stimulation filter for extracting the involuntary muscle contraction signal by performing a pre-processing operation to remove the noise signal or the distortion included in the multi-frequency impact response signal m-FIRS, and a feature extraction unit for extracting the feature vector related to muscle strength or muscular endurance based on the involuntary muscle contraction signal provided from the electrical stimulation filter.

In this embodiment, the feature vector in the time domain includes at least one of a feature used in a specific muscle diagnostic equipment, an envelope feature, a waveform pattern and shape, and a level crossing rate, and wherein the feature vector in the frequency domain includes at least one of a Percentile of Spectral Cumulative Sum (PoSCS), a Log Power Spectrum, a Percentile Pattern of Spectral Cumulative Sum (PPOSCS), and a log power spectrum shift.

In this embodiment, the feature used in a specific muscle diagnostic equipment includes at least one of a muscle tone state, a stiffness of a muscle, a decrement indicating the elasticity of the muscle, a relaxation time of the muscle, and a creep of the muscle.

In this embodiment, the artificial intelligence model learning unit includes a deep learning model using at least one of an initialization method of a random initialization method, a fine tuning of a backpropagation method, and an optimization algorithm of an adaptive moment estimation Adam, a cost function of Minimum Mean Square Error MMSE, an active function of an exponential linear unit ELU.

According to an embodiment, an electrical stimulation treatment system for controlling and generating a functional electrical stimulation signal by collecting an electromyography signal generated in response to electrical stimulation from a body, the system comprises, a voluntary/involuntary muscle contraction detection unit that extracts a feature vector from the frequency domain of the electromyography signal and distinguishes and detects a voluntary muscle contraction signal and an involuntary muscle contraction signal from the extracted feature vector by applying an artificial intelligence model, an involuntary muscle contraction signal removal unit that removes the involuntary muscle contraction signal from the electromyography signal according to the detection result, a muscle activity intensity calculator for calculating a root mean square RMS of the electromyography signal from which the involuntary muscle contraction signal is removed, and a functional electrical stimulation control unit that compares the effective value with a threshold value and generates the functional electrical stimulation signal to be applied to the body according to the comparison result.

In this embodiment, the feature vector includes at least one of a percentile of spectral cumulative sum PoSCS and a log power spectrum detected in the frequency domain of the electromyography signal.

In this embodiment, the involuntary muscle contraction signal removal unit attenuates the section including the involuntary muscle contraction signal of the electromyography signal by 6 dB to remove the involuntary muscle contraction signal.

In this embodiment, the artificial intelligence model distinguishes the involuntary muscle contraction signal and the voluntary muscle contraction signal from the electromyography signal by using an artificial intelligence algorithm.

In this embodiment, the involuntary muscle contraction signal removal unit comprises, a window unit for selecting a window of the electromyography signal, a fast Fourier transform unit for processing a signal included in the selected window by fast Fourier transform, a magnitude and phase calculator for calculating magnitudes and phases of signals output from the fast Fourier transform unit, respectively, a peak detector for detecting a peak in the magnitude of the signal, and a peak removing unit for filtering a noise signal corresponding to the detected peak.

### BRIEF DESCRIPTION OF THE FIGURES

The above and other objects and features of the present disclosure will become apparent by describing in detail embodiments thereof with reference to the accompanying drawings.

FIG. 1 is a block diagram exemplarily showing a sarcopenia diagnosis system according to an embodiment of the present invention.

FIGS. 2a to 2b exemplarily show the electrical stimulation and measurement unit shown in FIG. 1.

FIG. 3 is a block diagram for exemplarily explaining the configuration and operation of the response signal analyzer shown in FIG. 1.

FIG. 4a is a graph exemplarily showing the multi-frequency impact response signal m-FIRS shown in FIG. 3.

FIG. 4B is a waveform diagram showing the function of the electrical stimulation filter ESS shown in FIG. 3.

FIGS. 5a to 5a are diagrams illustrating examples of time domain feature extraction by the feature extractor shown in FIG. 3.

FIGS. 6a to 6d are diagrams illustrating examples of frequency domain feature extraction by the feature extraction unit illustrated in FIG. 3.

FIG. 7 is a flowchart exemplarily illustrating an operation method of the sarcopenia diagnosis system shown in FIG. 1.

FIG. 8 is a diagram for exemplarily explaining step S230 of the sarcopenia diagnosis system shown in FIG. 7.

FIG. 9 is a graph exemplarily showing a torque measuring device and measurement data for obtaining reference data for analyzing the sarcopenia diagnostic effect of the present invention.

FIGS. 10 and 11 are graphs and tables briefly showing the experimental results of FIG. 9.

FIG. 12 is a block diagram exemplarily showing an electrical stimulation treatment system according to an embodiment of the present invention.

FIG. 13 is a block diagram exemplarily showing the configuration of the electrical stimulation treatment system of FIG. 12.

FIG. 14 is a flowchart illustrating a processing method in a frequency domain for extracting a percentile of spectral cumulative sum PoSCS as an example of feature extraction.

FIG. 15 is a graph showing a method of extracting a cumulative sum of percentile spectra PoSCS.

FIG. 16 is a probability density function PDF showing the results of extracting the percentile of spectral cumulative sum PoSCS for each frequency from the EMG signal.

FIG. 17 is a flowchart illustrating a learning method of an artificial intelligence calculating unit for separating a voluntary muscle contraction signal and an involuntary muscle contraction signal according to an embodiment of the present invention.

FIG. 18 is a diagram schematically showing the structure of an LSTM algorithm for discriminating a voluntary muscle contraction signal and an involuntary muscle contraction signal through sequential electromyography EMG data in the time domain of the present invention.

FIG. 19 is a flowchart showing an actual operation and a test operation of the voluntary/involuntary muscle contraction detecting unit of the present invention.

FIG. 20 is a block diagram exemplarily showing the involuntary muscle contraction signal removal unit shown in FIG. 13.

FIGS. 21 and 22 are graphs showing results of frequency analysis of EMG data in the peak detector 2125 and the peak suppressor 1126.

FIG. 23 is a graph showing a waveform with a pre-processing process in the inverse transform unit and a waveform without a pre-processing process.

FIGS. 24 to 25 are diagrams showing the results of testing the performance of the electrical stimulation treatment system for generating functional electrical stimulation FES based on the muscle stimulation signal of the present invention.

### DETAILED DESCRIPTION

Hereinafter, embodiments of the present invention will be described clearly and in detail to the extent that those skilled in the art can easily practice the present invention.

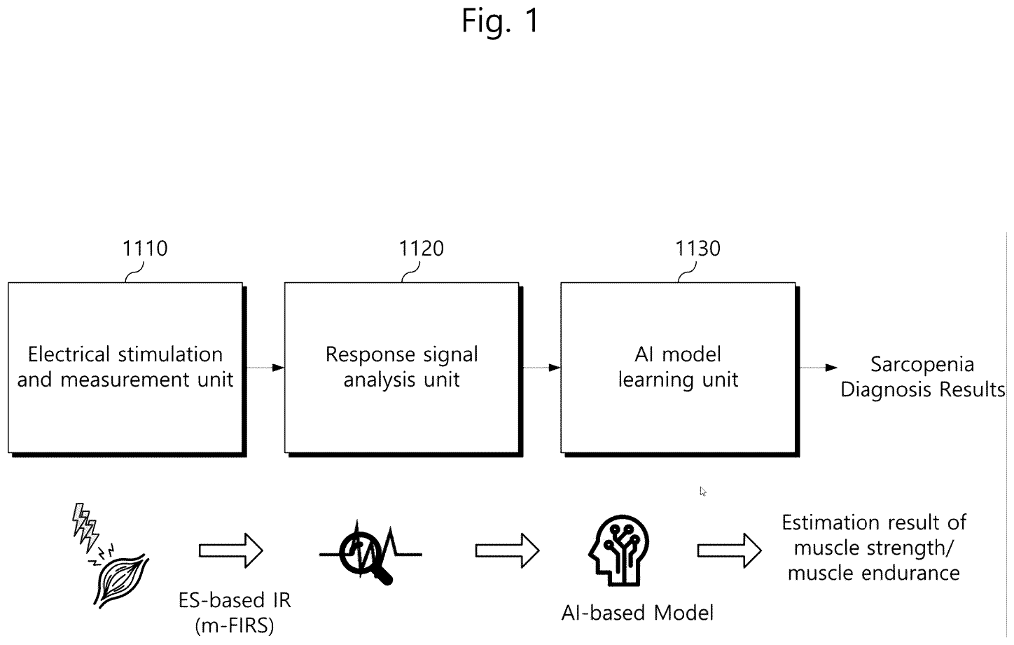

FIG. 1 is a block diagram exemplarily showing a sarcopenia diagnosis system according to an embodiment of the present invention. Referring to FIG. 1, a sarcopenia diagnosis system 1100 may include an electrical stimulation and measurement unit 1110, a response signal analysis unit 1120, and an artificial intelligence (hereinafter, AI) model learning unit 1130.

The electrical stimulation and measurement unit 1110 may be connected to the response signal analysis unit 1120 by wire or wirelessly. The electrical stimulation and measurement unit 1110 applies electrical stimulation (hereinafter, ES) to body muscles such as leg muscles, back muscles, and pectoral muscles. And then, the electrical stimulation and measurement unit 1110 measures an Electrical Stimulation-based Impact-pulse Response Signal (hereinafter, ES-based IR) and provides the measured value to the response signal analysis unit 1120. Here, the electrical stimulation-based 1 (ES-based IR) may mean electromyography EMG data obtained while applying electrical stimulation to the muscle. The EMG data may include EMG data measured by an EMG sensor. In particular, in the present invention, the electrical stimulation applied to the muscle is provided as multi-frequency electrical stimulation. Accordingly, the EMG data may be provided as a multi-frequency impact response signal (hereinafter, m-FIRS). Electromyography EMG data is then provided to the response signal analysis unit 1120 1120 through a preprocessing process that removes the electrical stimulation signal and minimizes distortion of involuntary muscle contraction components.

The response signal analysis unit 1120 may receive an electrical stimulation-based response signal (ES-based IR) from the electrical stimulation and measurement unit 1110 and analyze the response signal. The response signal analysis unit 1120 may remove a noise electrical signal included in the electrical stimulation-based response signal (ES-based IR). A reference signal for learning and performance evaluation of the artificial intelligence model may be measured through a torque equipment for measuring muscle strength and muscular endurance.

Also, the response signal analysis unit 1120 may extract a feature vector representing the characteristics of muscle strength and muscular endurance from the electrical stimulation-based response signal (ES-based IR). In addition, the response signal analysis unit 1120 may provide the extracted feature vector to the AI model learning unit 1130.

The AI model learning unit 1130 may receive a feature vector from the response signal analysis unit 1120. The AI model learning unit 1130 may perform AI model learning, such as deep learning or a support vector machine SVM. The AI model learning unit 1130 may generate a deep learning model and process a feature vector using the deep learning model. The AI model learning unit 1130 may classify the degree of muscle strength and muscular endurance based on the feature vector. The AI model learning unit 1130 may automatically find a relationship between the data provided from the response signal analysis unit 1120 and the diagnosis of sarcopenia through AI-based model learning. Therefore, the well-trained AI model learning unit 1130 may accurately predict and inform the sarcopenia diagnosis result (muscular strength or muscular endurance, etc.) corresponding to the input data.

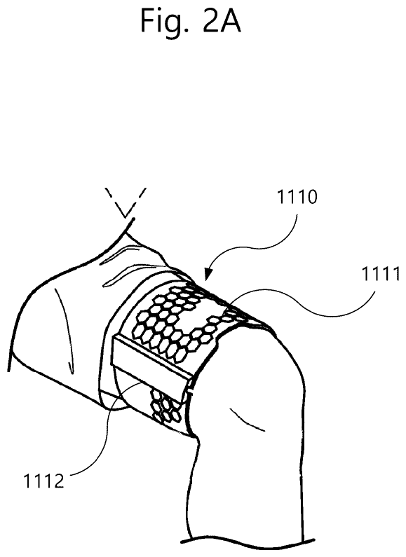

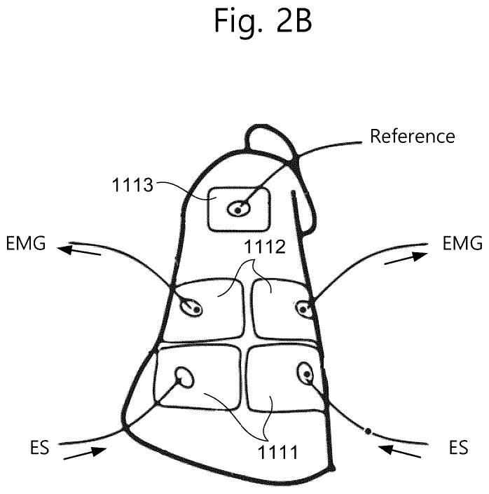

FIGS. 2A to 2B exemplarily show the electrical stimulation and

measurement unit shown in FIG. 1. The electrical stimulation and measurement unit 1110 may be variously implemented in the form of a belt or a pad. FIG. 2A shows the electrical stimulation and measurement unit 1110 in the form of a belt, FIG. 2B shows the electrical stimulation and measurement unit 1110 in the form of a pad exemplarily. The electrical stimulation and measurement unit 1110 may be worn on the user's body (e.g., thigh). The electrical stimulation and measurement unit 1110 may apply electrical stimulation ES to a user's body muscle (e.g., thigh muscle) and measure a response signal.

Referring to FIG. 2A, the electrical stimulation and measurement unit 1110 may include an electrical stimulation unit 1111 and an electrical stimulation measurement unit 1112. The electrical stimulation unit 1111 may include a stimulation signal generating circuit (not shown). The electrical stimulation unit 1111 may apply the electrical stimulation ES to the thigh using the stimulation signal generating circuit. The electrical stimulation unit 1111 may apply electrical stimulation ES to the user's muscles in order to collect the user's bio-signals (e.g., EMG signals).

The stimulation signal generating circuit may generate a signal for electrical stimulation ES. The stimulation signal generating circuit may include an ES generator for applying electrical stimulation to the thigh muscle. The electrical stimulation unit 1111 may apply the electrical stimulation signal generated by the ES generator to the thigh muscle using the thigh electrical stimulation pad. The strength, frequency, current or waveform of the electrical stimulation signal may be adjusted according to the degree of muscle stimulation of the user. Here, the electrical stimulation applied to the muscle is provided as a multi-frequency electrical stimulation.

The electrical stimulation measurement unit 1112 may include a muscle measurement sensing circuit (not shown). The muscle measurement sensing circuit may be an electromyography (EMG) measurement sensing circuit. The muscle measurement sensing circuit may include an EMG sensor for sensing the thigh electromyography measurement. When the electrical stimulation applied to the muscle is a multi-frequency electrical stimulation, EMG data measured by the EMG sensor may be provided as a multi-frequency impact response signal m-FIRS. Meanwhile, the electrical stimulation measuring unit 1112 may provide measurement information (i.e., ES-based IR) to the response signal analyzing unit 1120.

In the electrical stimulation and measurement unit 1110, an electrode for applying an electrical stimulation and an electrode for sensing a response to the electrical stimulation may be arranged in an array form. The electrical stimulation and measurement unit 1110 may select a position of measure an electromyography (EMG) signal through an array-type electrode or a position to transmit an electrical stimulation signal to issue a command.

Referring to FIG. 2B, the electrical stimulation and measurement unit 1110 may be implemented in the form of a pad. The electrical stimulation unit 1111 may include an electrical stimulation pad. The electrical stimulation pad may be used in a wet form for single-use or multi-use. Alternatively, the electrical stimulation pad may be manufactured using a dry high-adhesive material in order to transmit a user's biological signal or an electrical stimulation signal of the innervation muscle. For example, the electrical stimulation pad may be manufactured as a conductive dry adhesive electrode pad using a carbon nano material.

The electrical stimulation measurement unit 1112 may use an electrical stimulation measurement pad, and the muscle measurement sensing circuit may be an electromyography (EMG) measurement sensing circuit. The muscle measurement sensing circuit may include an EMG sensor for sensing the thigh electromyography measurement. Meanwhile, the electrical stimulation measuring unit 1112 may provide measurement information (i.e., m-FIRS) to the response signal analyzing unit 1120. In addition, the electrical stimulation and measurement unit 1110 may include a reference measurement unit 1113. The reference electrode 1113 is an electrode for providing a ground level of the electrical stimulation unit 1111 or the electrical stimulation measurement unit 1112.

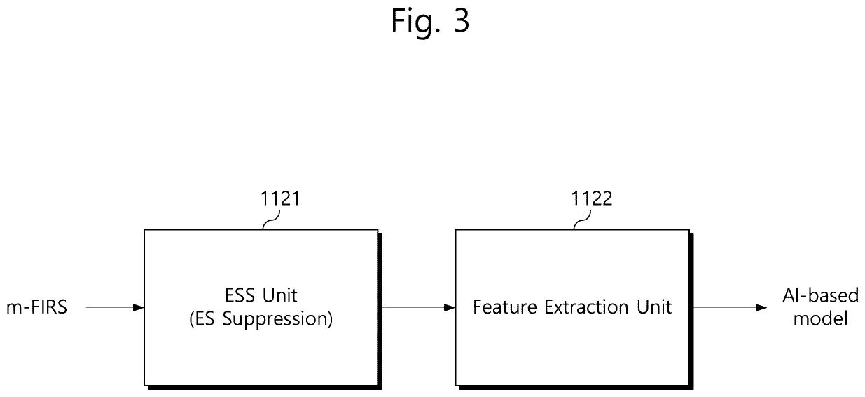

FIG. 3 is a block diagram for exemplarily explaining the configuration and operation of the response signal analyzer shown in FIG. 1. Referring to FIG. 3, the response signal analysis unit 1120 may include an electrical stimulation suppression (hereinafter, ESS) Unit 1121 and a feature extraction unit 1122. The response signal analysis unit 1120 may receive an electrical stimulation-based response signal (ES-based IR) from the electrical stimulation and measurement unit 1110 and perform data analysis.

The electrical stimulation-based response signal (ES-based IR) may refer to electromyography EMG data obtained when an electrical stimulation is applied to a muscle. The response signal analysis unit 1120 may analyze more various information when multi-frequency electrical stimulation is applied rather than single frequency electrical stimulation. Here, it is assumed that the electrical stimulation-based response signal (ES-based IR) is a multi-frequency impulse response signal m-FIRS.

Continuing to refer to FIG. 3, the ESS unit 1121 may receive a multi-frequency impact response signal m-FIRS. The ESS unit 1121 may remove the noise electrical signal included in the m-FIRS. The multi-frequency impact response signal m-FIRS includes an electrical stimulation signal applied from the electrical stimulation unit 1111 (see FIG. 3A) in addition to the involuntary muscle contraction signal, and the electrical stimulation signal has nonlinearity. Therefore, since the multi-frequency impact response signal m-FIRS includes data in the form of noise that differs according to skin and person, it is necessary to remove it.

The ESS unit 1121 removes the electrical stimulation signal included in the multi-frequency impact response signal m-FIRS and may undergo a pre-processing process to minimize distortion of the involuntary muscle contraction signal. It is possible to remove the electrical stimulation signal from the multi-frequency impact response signal m-FIRS and at the same time extract the involuntary muscle contraction signal with minimal distortion. The ESS unit 1121 may then perform signal processing to enable a more accurate analysis by a feature extraction unit 1122. For example, the ESS unit 1121 may perform a preprocessing operation applying a 5th order averaging filtering to 16 samples after the moment when the electrical stimulation is applied to remove the electrical stimulation signal and to reduce distortion. The output signal from the ESS unit 1121 may be expressed by the following equation.

$$y ( t + i ) = \{ x ( t + i - 2 ) + s ( t + i - 1 ) + s ( t + i ) + s ( t + i + 1 ) + s ( t + i + 2 ) \} / 5$$

Here, 1≤i≤15. ‘x’ is the input signal, ‘y’ is the output signal from which the electrical stimulation has been removed, ‘t’ is the time index indicating the moment of electrical stimulation output, and ‘i’ indicates the loop index.

The feature extraction unit 1122 may extract a feature vector related to muscle strength or muscular endurance based on a signal provided from the ESS unit 1121. For example, the feature extraction unit 1122 may extract time domain feature, such as characteristics used in ‘MyotonPro’, an envelope, a waveform pattern and shape, and a level crossing rate (LCR: Level Crossing Rate) from the involuntary muscle contraction signal. In addition, the feature extraction unit 1122 may extract a frequency domain feature such as a PoSCS (Percentile of Spectral Cumulative Sum) or Log Power Spectrum (hereinafter LPS), PPoSCS (Percentile Pattern of Spectral Cumulative Sum), and a log power spectrum LPS variation from the involuntary muscle contraction signal. Significant features are extracted from the multi-frequency impact response signal m-FIRS by the feature extraction unit 1122. The data extraction result of the feature extraction unit 1122 may be provided to the AI model learning unit 1130.

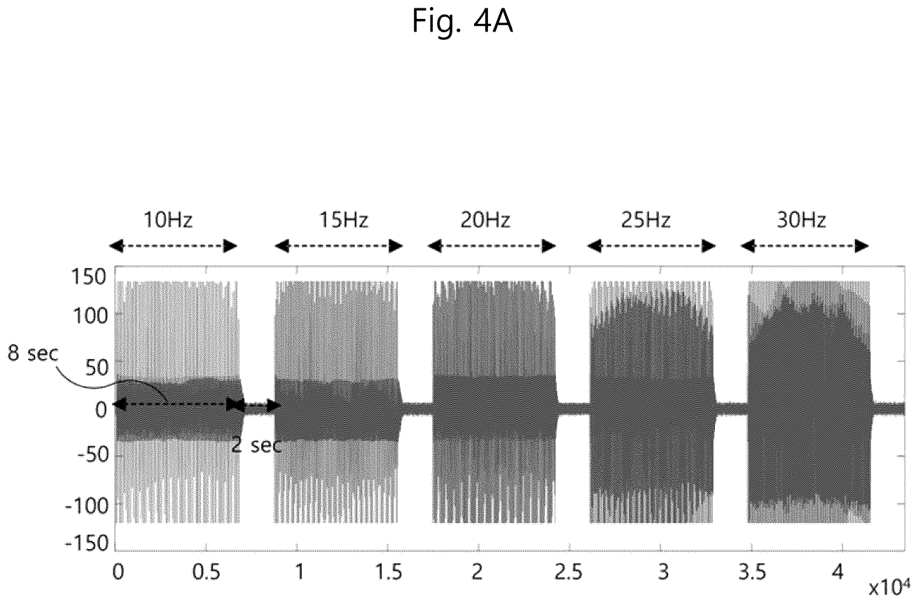

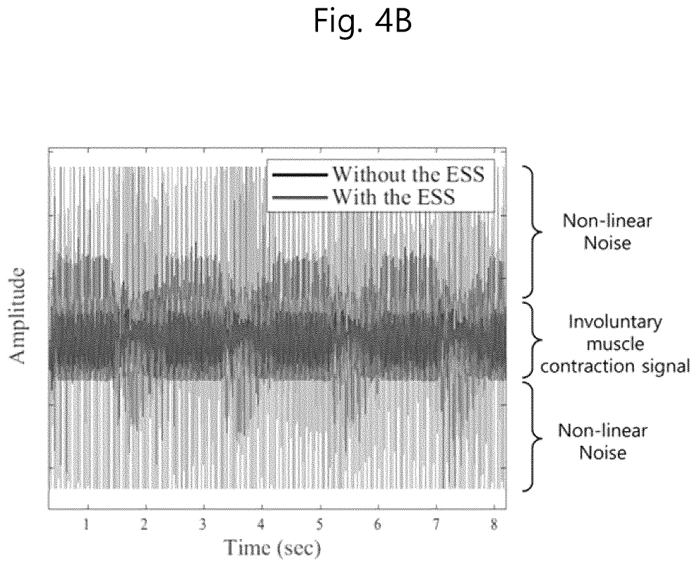

FIG. 4A is a graph exemplarily showing the multi-frequency impact response signal m-FIRS shown in FIG. 3. The electrical stimulation-based response signal (ES-based IR) is a signal serving as a basis for classifying the quality of a muscle by observing and analyzing a change in a muscle that responds as the frequency of an electrical stimulation changes.

Referring to FIG. 4A, a multi-frequency impact response signal m-FIRS is a muscle stimulation (EMG) signal obtained by applying a multi-frequency electrical stimulation to a muscle. FIG. 4A shows an example in which electrical stimulation is applied for 8 seconds in the order of 10 Hz, 15 Hz, 20 Hz, 25 Hz, and 30 Hz. The time interval of the rest period between each frequency is 2 seconds. After 30 Hz, the peak-to-peak distance of electrical stimulation becomes too narrow, and the component for involuntary contraction may be excessively reduced. Therefore, the response signal analysis unit 1120 may collect and measure the multi-frequency impact response signal m-FIRS blew 30 Hz. In addition, during feature extraction, data of a period corresponding to the rest period is removed, and thus the total size of data means the entire data length excluding the rest period. Here, 10 Hz, 15 Hz, 20 Hz, 25 Hz, and 30 Hz have been described as examples of multi-frequency, but the present invention is not limited to the frequencies disclosed herein. It will be well understood that multi-frequency at various intervals in various frequency ranges may be used to obtain a multi-frequency impact response signal m-FIRS.

FIG. 4B is a waveform diagram showing the function of the electrical stimulation filter (ESS) shown in FIG. 3, Referring to FIG. 4B, before the multi-frequency impact response signal m-FIRS is processed by the ESS unit 1121, it may be observed as a black waveform including a non-linear noise portion such as an electrical stimulation signal. However, when these nonlinear noises are suppressed or removed by the pre-processing operation by the ESS unit 1121, the multi-frequency impact response signal m-FIRS includes only a waveform with minimal distortion including a red involuntary muscle contraction signal.

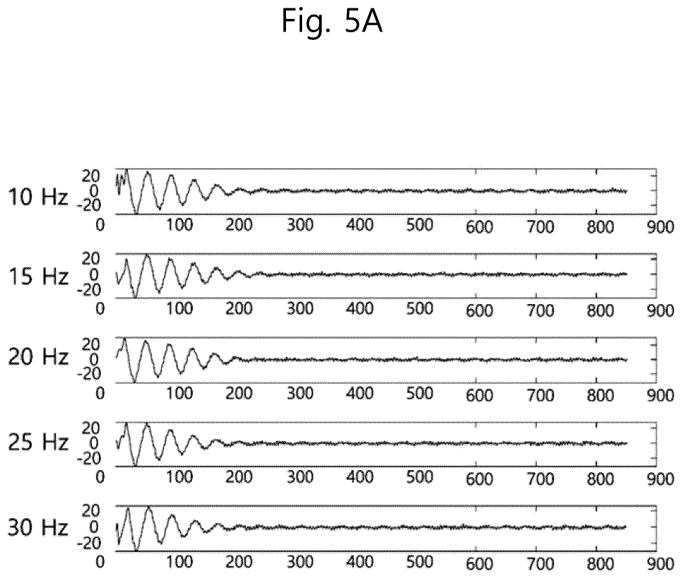

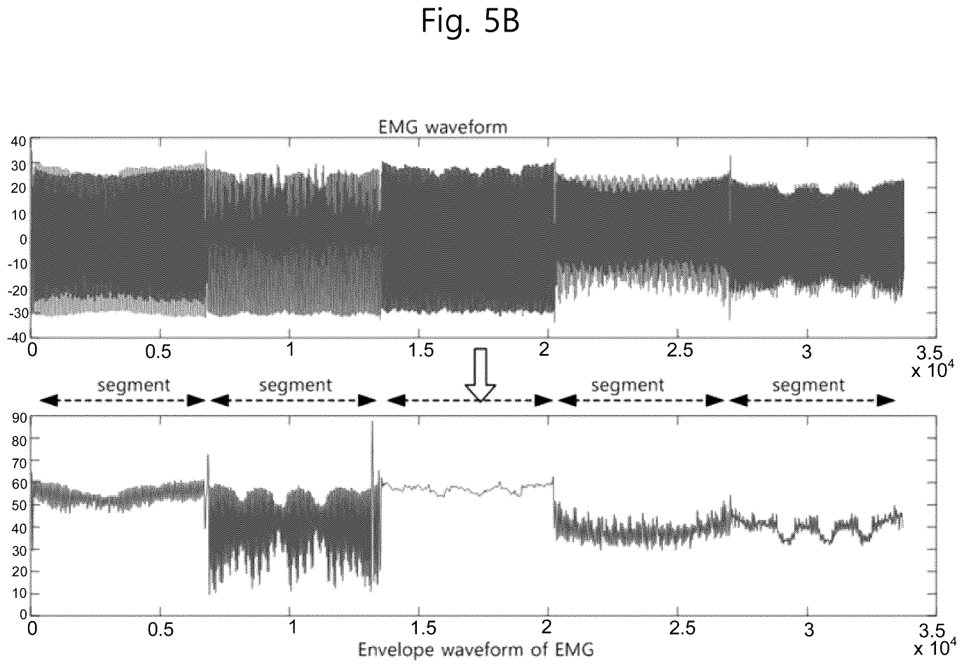

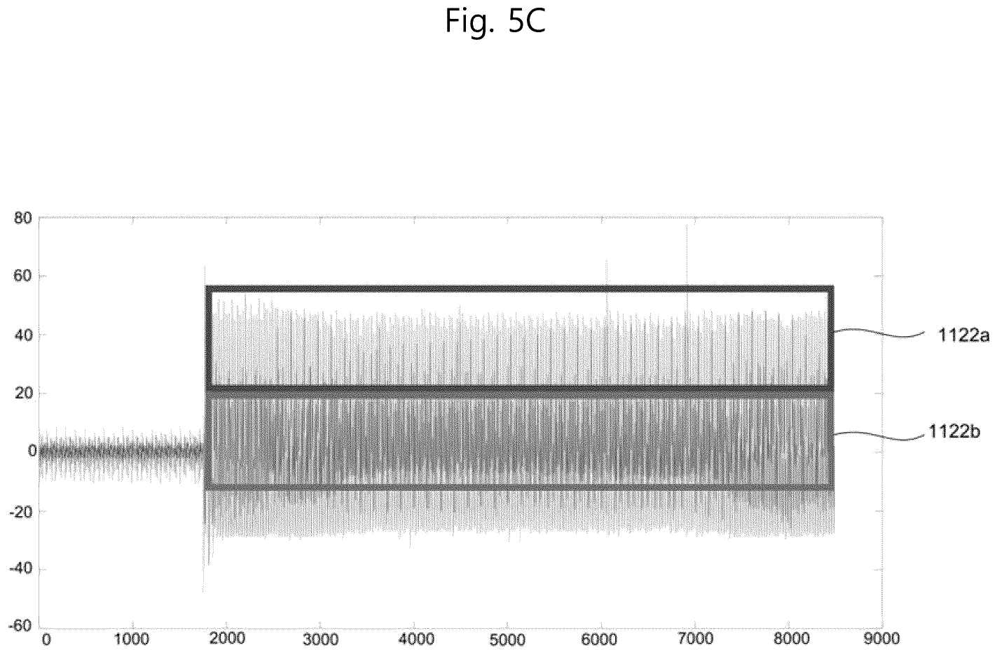

FIG. 5A to FIG. 5C are diagrams illustrating examples of time domain feature extraction by the feature extractor shown in FIG. 3. FIG. 5A shows a method of extracting feature used in ‘MyotonPro’, a portable muscle diagnosis device, using residual signals obtained after electrical stimulation. FIG. 5B shows a method for extracting envelope characteristics from involuntary muscle contraction signals. FIG. 5C is a waveform diagram exemplarily illustrating a method of extracting a level crossing rate (LCR) from an involuntary muscle contraction signal.

Referring to FIG. 5A, features similar to ‘MyotonPro’ may be extracted after the electrical stimulation provided by the electrical stimulation and measurement unit 1110 of FIG. 1. For example, in the electrical stimulation and measurement unit 1110 may extract waveforms of the EMG signal generated after applying the electrical stimulation for a predetermined time (e.g., 8 seconds) in the order of 10 Hz, 15 Hz, 20 Hz, 25 Hz, and 30 Hz. Here, although not shown, the electrical stimulation and measurement unit 1110 of the present invention can extract features of ‘MyotonPro’, a muscle tone (Muscle Tone), muscle stiffness, vibration damping rate (Decrement) indicating muscle elasticity, muscle recovery time (Relaxation time), and muscle strain rate (Creep) through the electrical stimulation-based response signal (ES-based IR). Referring to FIG. 5B, a method of extracting an envelope from an electromyography signal is briefly shown. To extract envelope, positive peaks and negative peaks in an electromyography signal excluding rest period are interpolated and the difference between the positive and negative peaks. The envelope of the electromyography signal means the flow of the amplitude that the muscle vibrates by electrical stimulation. The mean, standard deviation, kurtosis, and skewness can be extracted from each segment (in units of 8 seconds) of the envelope of the EMG signal.

FIG. 5C shows a method of extracting a level crossing rate (LCR) from an involuntary muscle contraction signal in the time domain. In the level crossing rate LCR, in addition to the zero crossing rate ZCR that can check how fast the signal changes, it is possible to extract the crossing rate for each level to know how fast the signal vibrates according to each amplitude level.

Referring back to FIG. 5C, after removing the DC value of each segment, the crossing rate for each of the ‘y’ values from 0 to 30 may be extracted while increasing the amplitude. The crossing rates of the levels of the two regions shown respectively show an involuntary muscle contraction signal 1122a vibrating with a large width and a fine muscle vibration signal 1122b vibrating with a small width, respectively. Also, it is possible to extract the characteristic of the time domain by extracting the ZCR for each segment and then calculating the variance for all segments.

Although not shown in FIG. 5A and FIG. 5C described above, a waveform pattern and shape WPS may be further included as a time domain feature extractable from the involuntary muscle contraction signal. That is, after taking the absolute value of the waveform for each segment, and after extracting the total as a characteristic, the variance for all segments can be obtained, or the total amount of vibration of the involuntary muscle contraction signal can be measured by calculating the sum of the amplitudes. In addition, after taking the absolute value for each segment, kurtosis and skewness may be extracted. In addition, distribution characteristics may be extracted by calculating the kurtosis and skewness of the waveform for each segment.

The following equation is an example of extracting time domain characteristics, and shows how to obtain a waveform pattern and shape WPS.

$$PP ( n ) = \sum \vert yn ( t ) \vert , for 1 \leq n \leq 5$$

Here, Σ is the sum from 0 to Tn. ‘n’ is an index for each frequency (10 Hz, 15 Hz, 20 Hz, 25 Hz, 30 Hz), and Tn represents the length of the input signal. That is, after the absolute value is overlaid on the waveform for each segment, the sum PP(n) may be calculated, and variance may be extracted for all segments. Power variance PV can be easily calculated by calculating the variance, and the kurtosis pattern KP and skewness pattern SP of the waveform for each segment can be obtained in a general way.

The following equation is another example of time domain feature extraction, and shows how to obtain a level crossing rate pattern (LCR Pattern: LP).

$$LP ( n ) = ( 1 / Tn ) \sum \{ s ( t ) - s ( t - 1 ) \} , for 1 \leq n \leq 5$$

$$s ( t ) = 1 , if ( yn ( t ) - α ) > 0$$

$$s ( t ) = 0 , if ( yn ( t ) - a ) \leq 0$$

Here, Σ is the sum from 0 to Tn. ‘α’ is a level crossing finite constant (Constant) and has a value between 1 and 30.

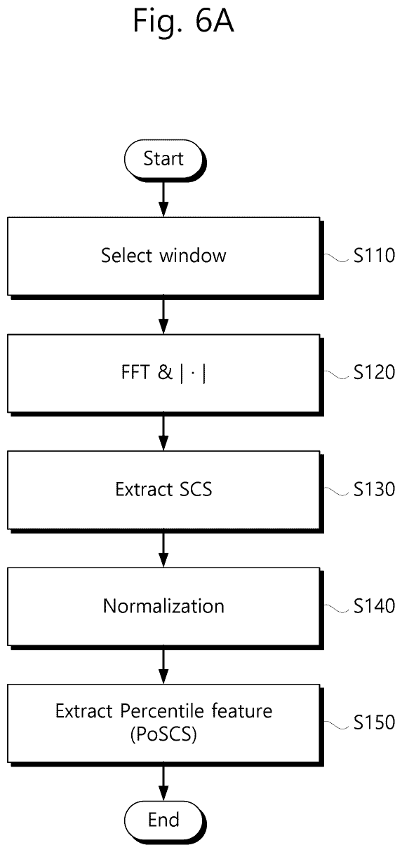

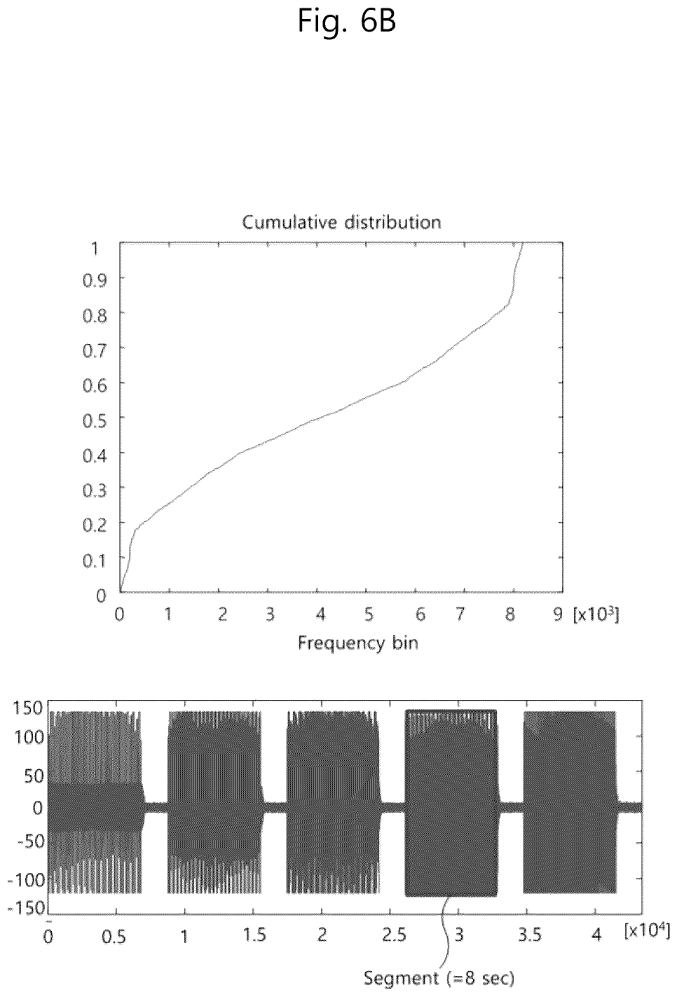

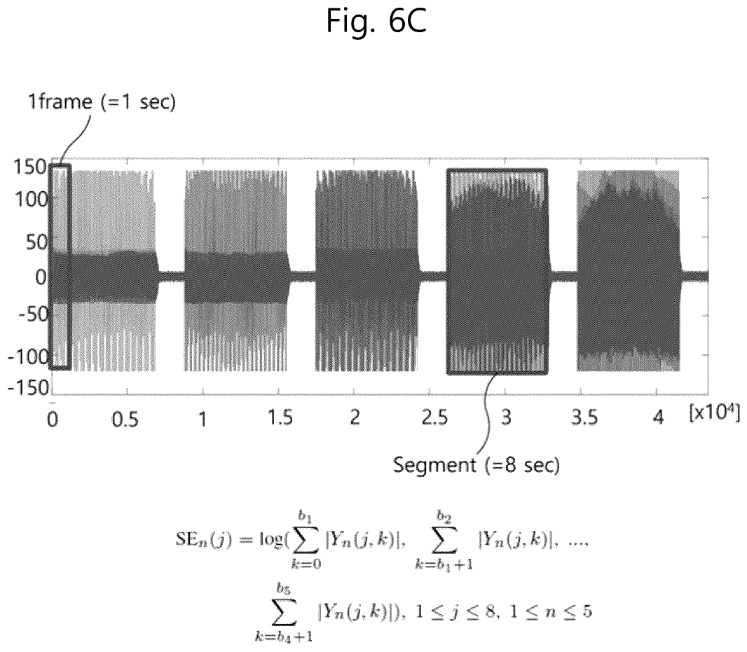

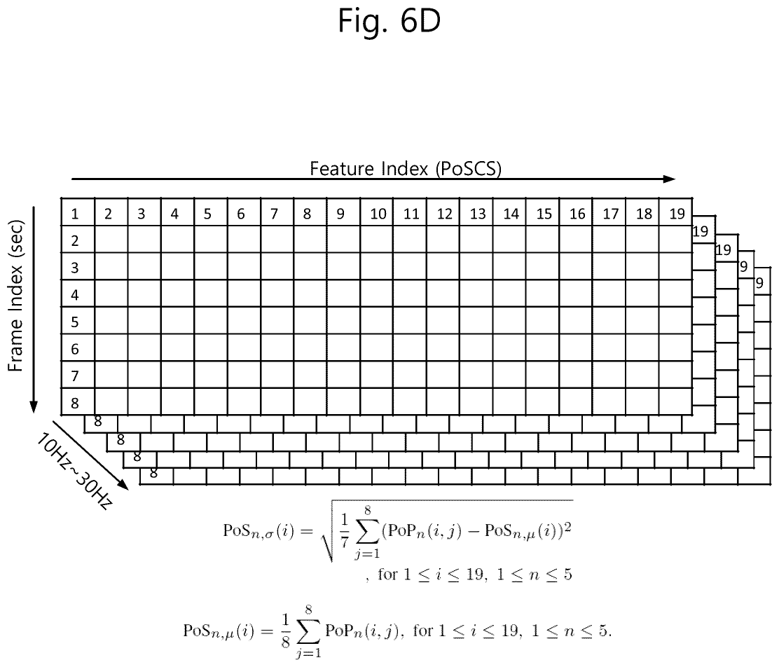

FIGS. 6A to 6D are diagrams illustrating examples of frequency domain feature extraction by the feature extraction unit illustrated in FIG. 3.

FIG. 6A is a flowchart schematically illustrating a procedure for extracting a characteristic in the frequency domain. Referring to FIG. 6A, a Percentile of Spectral Cumulative Sum (PoSCS) may be extracted as a frequency characteristic from a frequency component of an involuntary muscle contraction signal.

In step S110, a window in the time domain of the involuntary muscle contraction signal to be converted into a frequency spectrum is selected. For example, the window of the signal from which the rest period of the multi-frequency impact response signal m-FIRS is removed may be selected on a sector-by-sector basis or on a frame-by-frame basis. In step S120, fast Fourier transform FFT and absolute value calculation are performed on the window of the selected involuntary muscle contraction signal. In step S130, a spectral cumulative sum SCS is extracted in the frequency domain based on the absolute value calculation result. In step S140, a normalization operation is performed. In step S150, a Percentile of Spectral Cumulative Sum PoSCS of each of the frequencies is extracted based on the normalized data.

FIG. 6B is a graph for explaining a process of extracting a Percentile of Spectral Cumulative Sum PoSCS. The PoSCS feature extraction may be exemplarily performed through the following process. First, the magnitude is accumulated in the positive x-axis direction in the frequency domain, and then max-normalization data is used. Next, for each segment, characteristics are extracted in 5% units from 5 to 95%. In this case, the dimension may be 95*(5 segments). Next, for each segment, a ‘y’ value is calculated in a specific frequency bin(1 to 32, unit: 1). In this case, the dimension of the ‘y’ value may be 32*(5 segments). Next, two types of characteristics are additionally extracted for the entire waveform. In this case, the dimension may be 127.

The following equation is an example of frequency domain feature extraction, and shows how to obtain the Percentile of Spectral Cumulative Sum PoSCS.

$$PoSCSn ( i ) = arg min ( \vert fn - 0.01 i \vert ) , for 1 \leq i \leq 95 , 1 \leq n \leq 5$$

$$fn ( k ) = [ 1 / ( fn ( K - 1 ) ) ] \sum Yn ( m ) , for 1 \leq k < K , 1 \leq n \leq 5$$

Here, Σ is the sum of m=0 to k. ‘m’ is a frequency bin index, ‘i’ is a horizontal line index, fn(k) is a spectral cumulative sum function, and ‘K’ is a half value of the FFT size.

FIG. 6C is a graph for explaining a process of extracting a spectral band power envelope (hereinafter referred to as SE) by the feature extraction unit. The spectral band power envelope SE may be obtained by band-based extraction. The spectral band power envelope SE feature extraction may be exemplarily performed through the following process.

First, a segment is divided into frames(=1 second), and then features are extracted. In this case, the dimension may be 280. The FFT size for the spectral band power envelope SE is 1024, and 513 frequency bins including the DC component and the fold-over frequency may appear at 511, which is half the size. Utilizing all frequency bins as feature vectors may cause overfitting of the model. Therefore, in order to reduce the dimension, frequency bins are grouped into bands, then all are added and ‘log’ is applied. In this case, the reason for taking ‘log’ is to minimize the degradation of the model's performance due to the excessively wide range of values.

The equation shown in FIG. 6C is an example of frequency domain feature extraction, and shows a method of obtaining the spectral band power envelope SE. In the equation of FIG. 6c, b1, b2, . . . b7 indicates the frequency index of the bands.

FIG. 6D is a diagram schematically illustrating a matrix for extracting PoSCS-STAT(POS), which is one of the features in the frequency domain. Referring to FIG. 6D, a feature index (PoSCS) matrix may be generated for each of the five segments for each frequency, and the average and standard deviation may be obtained for each column. First, the feature index (PoSCS) for 8 frames in a segment may be extracted as a change amount of the feature index (PoSCS) to which the muscle responds every 1 second. And the mean and standard deviation for the entire matrix can be extracted.

The following equation is another example of frequency domain feature extraction, and shows how to obtain PoSCS-STAT (POS).

$$PoPn ( i , j ) = arg min ( \vert fn , j - 0.05 i \vert ) , for 1 \leq i \leq 19 , 1 \leq j \leq 8 , 1 \leq n \leq 5$$

The equation shown in FIG. 6D is another example of frequency domain feature extraction, and shows a method of obtaining PoSCS-STAT (POS). In FIG. 6D, ‘j’ denotes the frame index, ‘μ’ denotes the mean, and ‘σ’ denotes the standard deviation.

The following equations are other examples of frequency domain feature extraction, and are equations showing how to obtain the SBPE GAP(SG).

$$LPSD 1 = LPS 15 Hz - LPS 10 Hz$$

$$LPSD 2 = LPS 20 Hz - LPS 10 Hz$$

$$LPSD 3 = LPS 25 Hz - LPS 10 Hz$$

$$LPSD 4 = LPS 30 Hz - LPS 10 Hz$$

LPSD stands for log power spectral differential.

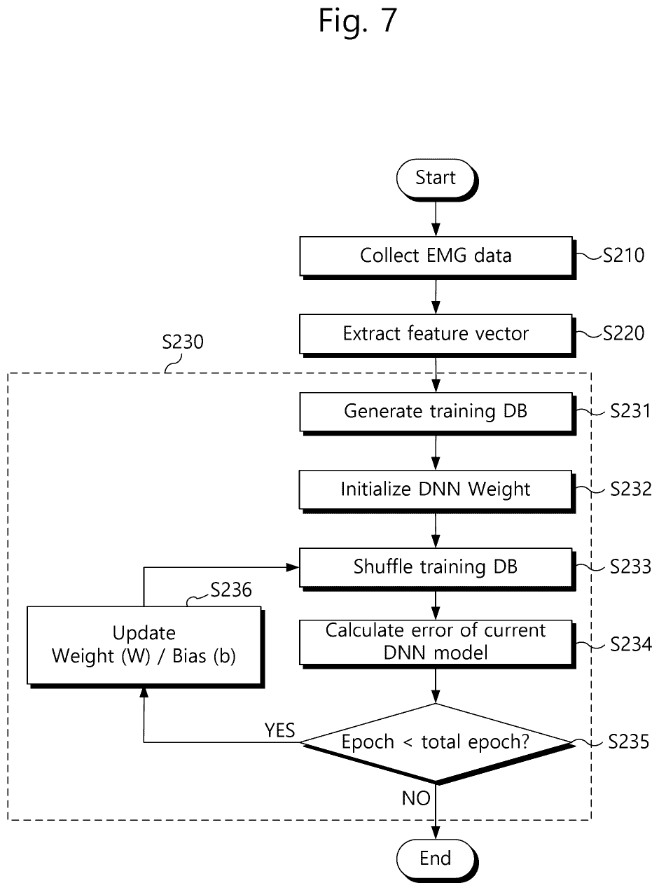

FIG. 7 is a flowchart exemplarily illustrating an operation method of the sarcopenia diagnosis system shown in FIG. 1. As described in FIG. 1, the sarcopenia diagnosis system 1100 may include an electrical stimulation and measurement unit 1110, a response signal analysis unit 1120, and an AI model learning unit 1130.

In step S210, the electrical stimulation and measurement unit 1110 may collect electromyography EMG data. The electrical stimulation and measurement unit 1110 may apply electrical stimulation ES to a body muscle and measure an electrical stimulation-based response signal ES-based IR. The electrical stimulation-based response signal ES-based IR may be a multi-frequency impact response signal m-FIRS obtained when electrical stimulation of multiple frequencies is applied to the muscle.

In step S220, the response signal analysis unit 1120 may analyze the multi-frequency impact response signal m-FIRS and extract a feature vector. The response signal analysis unit 1120 may remove a noise electrical signal included in the multi-frequency impact response signal m-FIRS and then extract a feature vector related to muscle strength or muscular endurance. In addition, the response signal analysis unit 1120 may provide the result of extracting the feature vector to the AI model learning unit 1130.

In step S230, the AI model learning unit 1130 may receive the feature vector from the response signal analysis unit 1120 and perform AI model learning. The AI model learning unit 1130 may find a correlation between the data extracted from the feature vector and the diagnosis of sarcopenia through AI-based model learning, and estimate the sarcopenia diagnosis result (muscle strength or muscular endurance, etc.).

The AI model learning unit 1130 may receive the feature vector and generate a database for learning (S231). The AI model learning unit 1130 may initialize a deep neural network (DNN) weight (S232). The AI model learning unit 1130 may shuffle the training database DB (S233). The AI model learning unit 1130 may calculate the current DNN model error (S234). The AI model learning unit 1130 determines whether the epoch learned so far is smaller than the last epoch (S235). The AI model learning unit 1130 terminates if the epoch learned so far is not small (NO direction), and if it is small (YES direction), updates the DNN weight and bias (S236), and performs step S233.

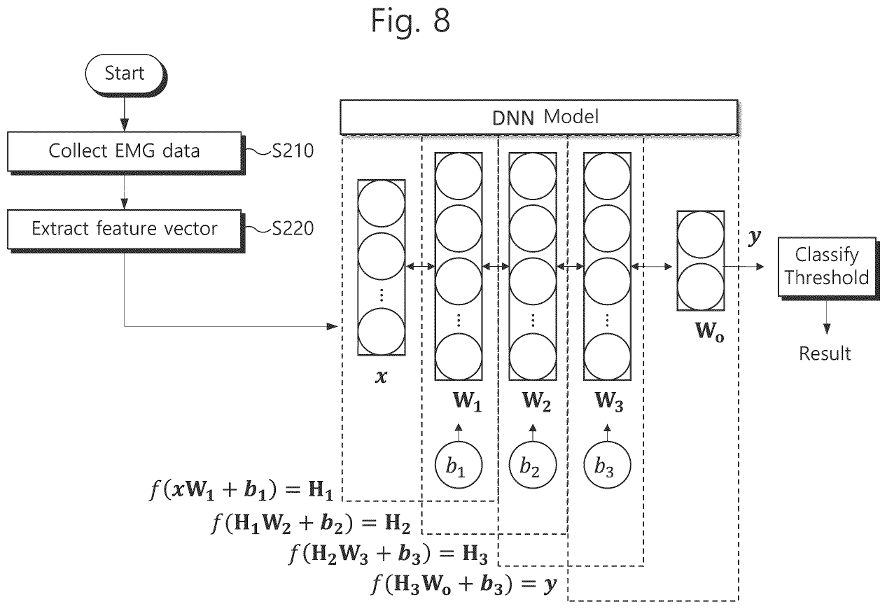

FIG. 8 is a diagram for exemplarily explaining step S230 of the sarcopenia diagnosis system shown in FIG. 7. In the functional formula of the DNN model shown in FIG. 8, ‘f’ means an activation function, ‘W’ means a weight parameter of the DNN, and ‘b’ means a bias parameter of the DNN.

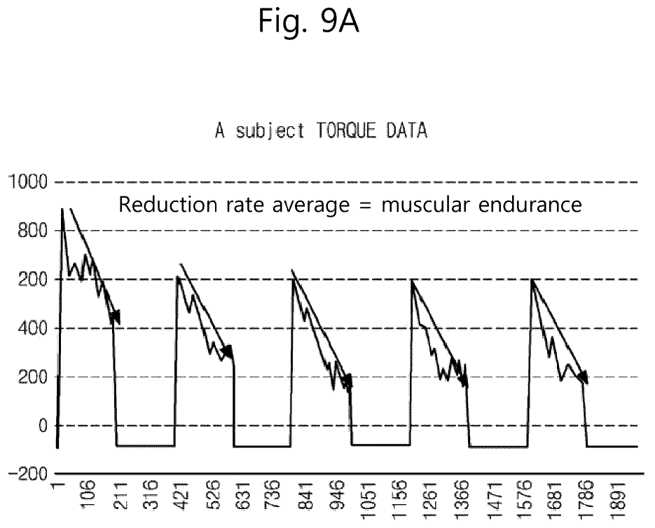

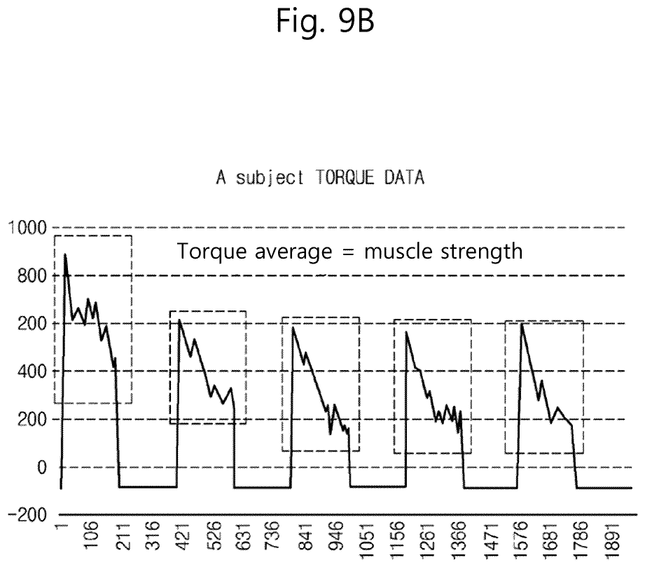

A DNN model may consist of an input layer, a hidden layer, and an output layer. An input layer receives an input value (x). In the hidden layer, weight parameters (W1, W2, W3) and bias parameters (b1, b2, b3) exist, and each step is performed according to the functional formula shown in FIG. 9. The first hidden layer outputs the first hidden value ‘H1’ using the input value ‘x’, the first weight ‘W’, and the first bias ‘b1’. The second hidden layer outputs the second hidden value ‘H2’ using the first hidden value ‘H1’, the second weight ‘W2’, and the second bias ‘b2’. Similarly, the third hidden layer outputs the third hidden value ‘H3’. The output layer finally outputs the output value ‘y’ using the third hidden value ‘H3’, the weight ‘W0’, and the third bias ‘b3’. The DNN model finally performs threshold classification using the output value ‘y’.

FIG. 9A and FIG. 9B are graphs exemplarily showing torque measurement data for obtaining reference data for analyzing the sarcopenia diagnostic effect of the present invention. Referring to FIG. 9A and FIG. 9B, the sarcopenia diagnosis system may obtain analysis data in the following experimental configuration.

First, electromyography EMG data is obtained using the sarcopenia diagnosis system of the present invention. Electromyography EMG data can be collected by changing the electrical stimulation from 10 Hz to 30 Hz in 5 Hz increments and measuring the electrical stimulation-based response signal (ES-based IR). For example, by collecting electromyography EMG data 5 times per person, it may be possible to extract features in the time domain or frequency domain described above.

Then, reference data is collected from those who collected electromyography EMG data using a torque measuring device. When measuring torque, apply force to the torque device as much as possible for 30 seconds without holding the chair. This is to apply force to the thigh as much as possible. This measurement routine can be performed 5 times after a 1-minute break. It is possible to measure muscular endurance through repeated measurements.

The graphs of FIGS. 9A and 9B show a method of extracting muscle strength and muscular endurance using a torque measuring device. First, the muscular endurance of FIG. 9A may be measured by measuring the average value of the reduction rate of the torque measured for 30 seconds five times. The initially measured torque value and the torque value measured after 30 seconds decrease as indicated by the arrow. The average of these reduction rates can be used as data for evaluating muscular endurance. And the muscle strength of FIG. 9B can be extracted by calculating the average value of the torque values measured 5 times for 30 seconds.

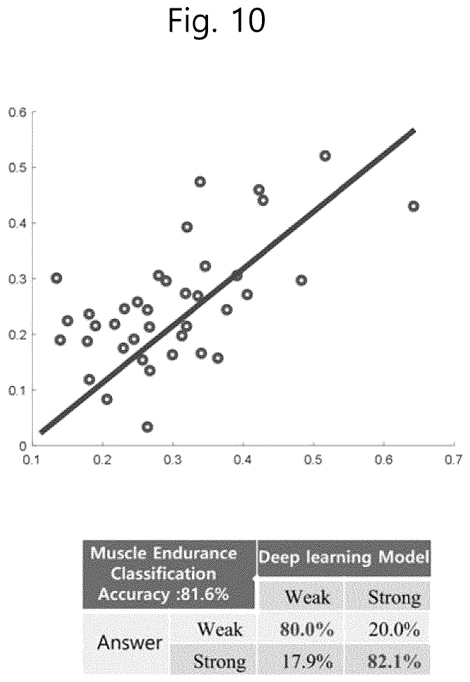

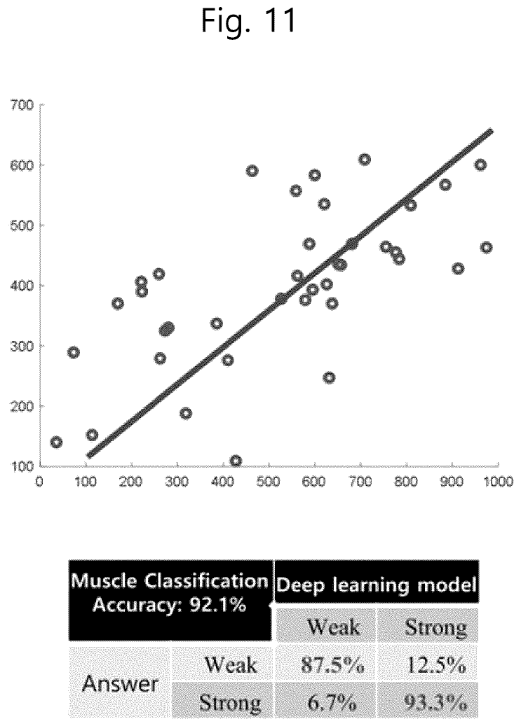

FIG. 10 and FIG. 11 are graphs and tables briefly showing the experimental results of FIG. 9. In setting the artificial intelligence AI model, among the extracted features, only characteristics having a correlation of ±0.3 and ±0.25 or more with respect to muscle strength and muscular endurance, respectively, can be selected as input. In addition, a random initialization method is applied to the initialization of the AI model, and an error backpropagation method can be applied to fine-tuning, and the number of hidden layers can be set to 3. And 32 hidden units may be set for each hidden layer. In addition, the adaptive moment estimation ADAM method is used as an optimization algorithm for determining the update method of weights. And the normalization of the 0.2, L1, and L2 layers can be applied to the dropout that deactivates the output node in the regularization method to prevent overfitting and apply model generalization. Minimum mean square error MMSE was used as a cost function, and an exponential linear unit (ELU) was used as an active function. As the output value of muscle strength, an output with a torque average of 500 or more (Torque average ≥500) was defined as strong (Class 1), and an output with a torque average of less than 500 (Torque average <500) was defined as weak (Class 2). In addition, as an output value of muscular endurance, a torque reduction rate of ‘0.3’ or more was defined as Class 1, and a torque reduction rate of less than ‘0.3’ was defined as Class 2.

Referring to FIG. 10, the experimental results for muscular endurance are shown. The upper graph shows the regression results for the estimated muscular endurance (horizontal axis) and the reference (vertical axis) measured using the actual torque equipment in the sarcopenia diagnosis system according to the present invention. The lower table shows the classification accuracy of muscle endurance by the deep learning model of the present invention.

Looking at the upper graph, muscle endurance values estimated according to an embodiment of the present invention and reference values measured using an actual torque device show a correlation of 0.61. This means that the muscle endurance value estimated using the deep learning model of the present invention has significant linearity with the actual muscle endurance.

Looking at the lower table, the output of the deep learning model of the present invention shows an accuracy of 80.0% when the muscular endurance is classified as a weak class, and an accuracy of 82.1% in the case of a strong class. Therefore, the classification accuracy of the deep learning model of the present invention for total muscular endurance is 81.6%.

Referring to FIG. 11, the experimental results for muscle strength are shown. The upper graph shows the regression results for the estimated muscle strength (horizontal axis) and the reference (vertical axis) measured using the actual torque equipment in the sarcopenia diagnosis system according to the present invention. The lower table shows the classification accuracy of muscle strength by the deep learning model of the present invention.

Looking at the upper graph, muscle endurance values estimated according to an embodiment of the present invention and reference values measured using an actual torque device show a correlation of 0.65. This means that the muscle strength estimated using the deep learning model of the present invention has significant linearity with the actual muscle strength.

Looking at the lower table, the output of the deep learning model of the present invention shows an accuracy of 87.5% when the muscular endurance is classified as a weak class, and an accuracy of 93.3% in the case of the strong class. Therefore, it can be confirmed that the classification accuracy of the deep learning model of the present invention for total muscular endurance is 92.1%.

Through the above experiments, the performance of the sarcopenia diagnosis system of the present invention was described. Through the setting of the deep learning model of the present invention, it is expected to be able to extract the features of muscle strength and muscular endurance with relatively high accuracy even with a simple method.

As described above, the sarcopenia diagnosis system according to an embodiment of the present invention shows positive aspect as a result of comparison and experimentation with a reference related to muscle strength and muscular endurance using an electrical stimulation-based response signal (ES-based IR). A feature based on an electrical stimulation-based response signal (ES-based IR) has a high correlation with a defined reference (strength/muscle endurance). According to the results of the experiment using the naive DNN model, the trend is well followed. Experimental results show that classification is possible to some extent. That is, it can be seen that the electrical stimulation-based response signal (ES-based IR) includes information corresponding to muscle strength and muscular endurance.

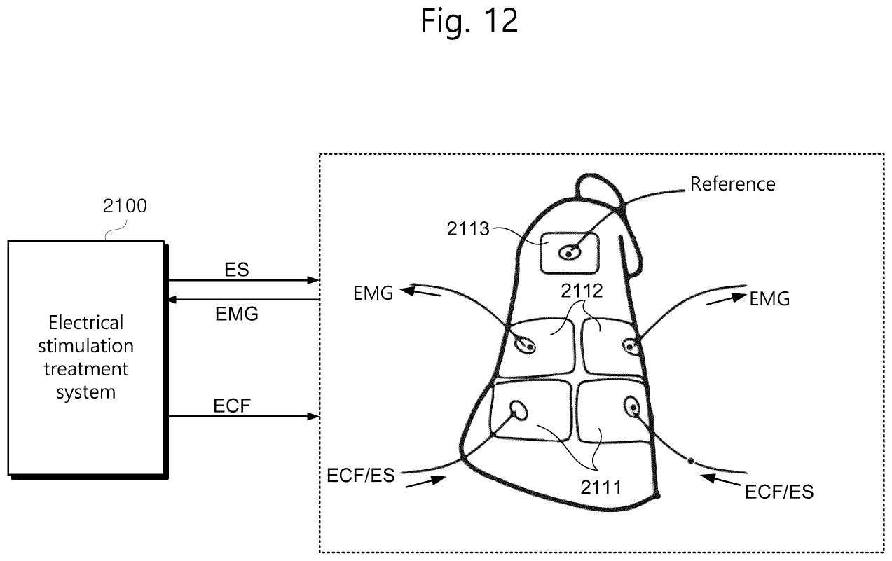

FIG. 12 is a block diagram exemplarily showing an electrical stimulation treatment system according to an embodiment of the present invention. Referring to FIG. 12, electrical stimulation treatment system 2100 pre-processes an electromyography signal EMG obtained by applying electrical stimulation, and generates functional electrical stimulation FES for treating a patient using the pre-processed data.

The electrical stimulation treatment system 2100 applies electrical stimulation ES to the muscle or skin of a patient, and generates functional electrical stimulation FES based on an electromyography signal EMG provided in response to the electrical stimulation ES. The electrical stimulation treatment system 2100 applies a preprocessing technique that separates and removes the involuntary muscle contraction signal from the electromyography signal EMG, unlike a general functional electrical stimulation FES signal. Of course, the collected electromyography signal EMG includes an electrical stimulation ES signal. The electrical stimulation treatment system 2100 extracts the voluntary muscle contraction signal by removing the electrical stimulation ES signal and the involuntary muscle contraction signal from the electromyography signal EMG, and generates functional electrical stimulation FES signal based on voluntary muscle contraction signals. Therefore, in that the functional electrical stimulation FES signal of the present invention is generated based on the electromyography signal EMG, it will be referred to as an electromyography-based functional electrical stimulation (ECF: EMG-Controlled FES) hereinafter.

The electrical stimulation treatment system 2100 may control functional electrical stimulation FES based on an electromyography signal EMG for measuring muscle activity according to muscle contraction. The electrical stimulation treatment system 2100 may adjust the strength of the electrical stimulation according to the root mean square RMS size of the electromyography signal EMG. Through this, the electrical stimulation treatment system 2100 can provide a rehabilitation treatment service in which the electrical stimulation is turned on when the force is applied above a certain level, and the electrical stimulation is turned off when the force falls below the predetermined level. In addition, when the user does not give the force for a specific operation, the electric stimulation treatment system 2100 may provide a service for applying electric stimulation to assist the insufficient force.

The electrical stimulation treatment system 2100 of the present invention uses electromyography-based functional electrical stimulation (ECF) to treat a patient. For this purpose, pad-type electrodes may be used. The electrical stimulation pad 2111 may include an electrical stimulation pad that applies electrical stimulation ES and functional electrical stimulation based on an electromyography signal ECF. For example, the electrical stimulation pad 2111 may be used in a wet form for single use or multiple uses. Alternatively, the electrical stimulation pad 2111 may be manufactured using a dry high-adhesive material to transmit a user's biological signal or an electrical stimulation signal of the innervation muscle. For example, the electrical stimulation pad 2111 may be manufactured as a conductive dry adhesive electrode pad using a carbon nano material. The electrical stimulation measurement pad 2112 is used for electromyography EMG measurement. The electrical stimulation measuring pad 2112 may include an EMG sensor for sensing the thigh electromyography. In addition, the reference pad 2113 is provided as an electrode pad for providing a ground level of the electrical stimulation pad 2111 or the electrical stimulation measurement pad 2112.

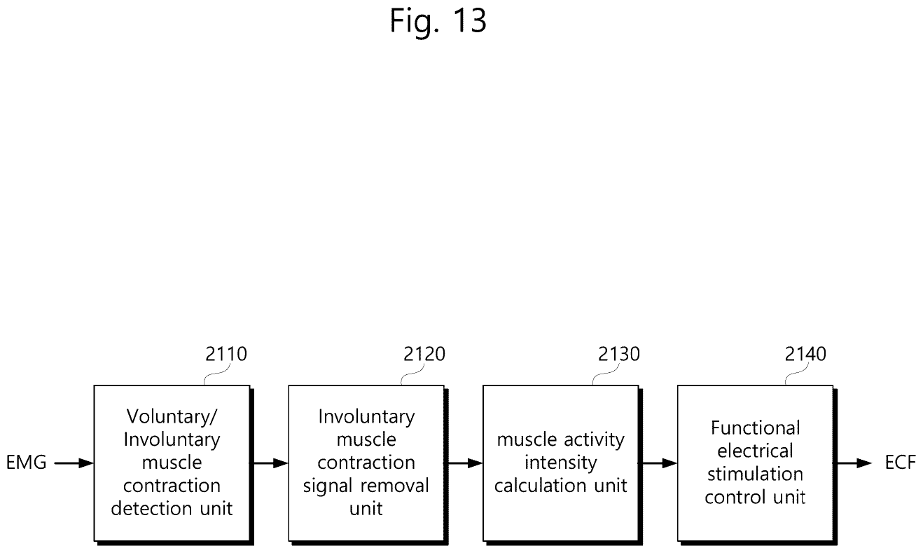

FIG. 13 is a block diagram exemplarily showing the configuration of the electrical stimulation treatment system of FIG. 12. Referring to FIG. 13, the electrical stimulation treatment system 2100 includes a voluntary/involuntary muscle contraction detection unit 2110, an involuntary muscle contraction signal removal unit 2120, a muscle activity intensity calculation unit 2130, and functional electrical stimulation control unit 2140.

The voluntary/involuntary muscle contraction detection unit 2110 receives an electromyography signal EMG collected in response to the electrical stimulation ES. The voluntary/involuntary muscle contraction detection unit 2110 may remove the electrical stimulation ES included in the input EMG signal, and distinguish the voluntary muscle contraction signal from the involuntary muscle contraction signal. It is difficult to distinguish between a voluntary muscle contraction signal and an involuntary muscle contraction signal only by the amplitude of the signal. Therefore, artificial intelligence AI models are needed to separate voluntary and involuntary muscle contraction signals.

For high-performance signal classification, in addition to high-performance deep learning models, high-performance feature vectors to improve the model's performance are also important. Therefore, in the present invention, a sampling rate of 850 Hz, a frame size of 320 samples, a shift size of 20 samples, and an FFT size of 512 may be applied for feature extraction. Since the size of the frame size is 320 samples, 320 samples will be sequentially stored in the buffer. And, after 320 samples have elapsed, signals may be sequentially stored in a buffer by 20 samples. After updating the buffer, feature vectors will be extracted using feature extraction techniques.

The involuntary muscle contraction signal removal unit 2120 removes the detected involuntary muscle contraction signal. The muscle activity intensity calculation unit 2130 calculates the RMS in a state in which the noise has been removed to intuitively grasp how much force is applied.

The functional electrical stimulation control unit 2140 generates electromyography-based functional electrical stimulation ECF. That is, the functional electrical stimulation control unit 2140 may turn on or off the application of the functional electrical stimulation by comparing the RMS with a specific threshold. For example, the functional electrical stimulation control unit 2140 may apply the functional electrical stimulation if the RMS is greater than or equal to the threshold value, and may not apply the functional electrical stimulation if the RMS is less than the threshold. Alternatively, the functional electrical stimulation control unit 2140 may determine the strength of the functional electrical stimulation according to the RMS. The functional electrical stimulation control unit 2140 may control the electrical stimulation to become stronger when the RMS is increased and to be weakened when the RMS is decreased.

The electrical stimulation treatment system 2100 may control functional electrical stimulation FES based on an electromyography signal EMG for measuring muscle activity according to muscle contraction. The electrical stimulation treatment system 2100 may adjust the strength of the electrical stimulation according to the RMS size of the EMG. Through this, the electrical stimulation treatment system 2100 can provide a rehabilitation treatment service in which the electrical stimulation is turned on when the force is applied above a certain level, and the electrical stimulation is turned off when the force falls below the predetermined level. In addition, when the user does not provide the force to be given for a specific operation, the electrical stimulation treatment system 2100 may provide a service that assists by applying electrical stimulation to assist the insufficient force.

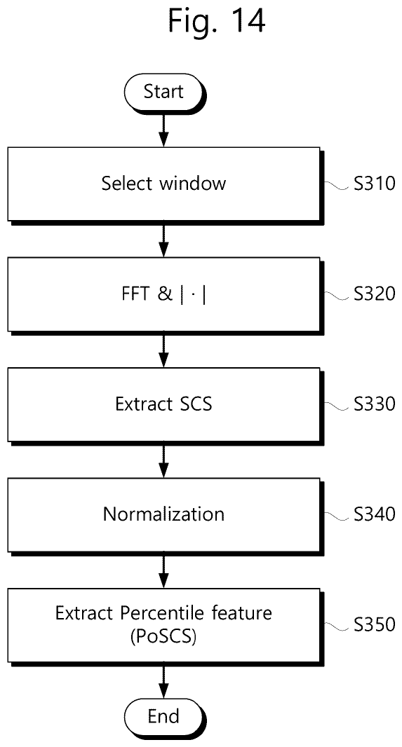

FIG. 14 is a flowchart illustrating a processing method in a frequency domain for extracting a Percentile of Spectral Cumulative Sum PoSCS as an example of feature extraction. Referring to FIG. 14, a Percentile of Spectral Cumulative Sum PoSCS may be extracted through frequency domain processing for the EMG signal.

In step S310, a window in the time domain of the EMG signal to be converted into a frequency spectrum is selected. For example, a window of the EMG signal EMG may be selected in units of sectors or frames.

In step S320, a fast Fourier transform FFT and absolute value calculation are performed on the window of the EMG signal of the selected section.

In step S330, a spectral cumulative sum SCS is extracted in the frequency domain based on the absolute value calculation result.

In step S340, a normalization operation is performed.

In step S350, a Percentile of Spectral Cumulative Sum PoSCS of each of the frequencies is extracted based on the normalized data.

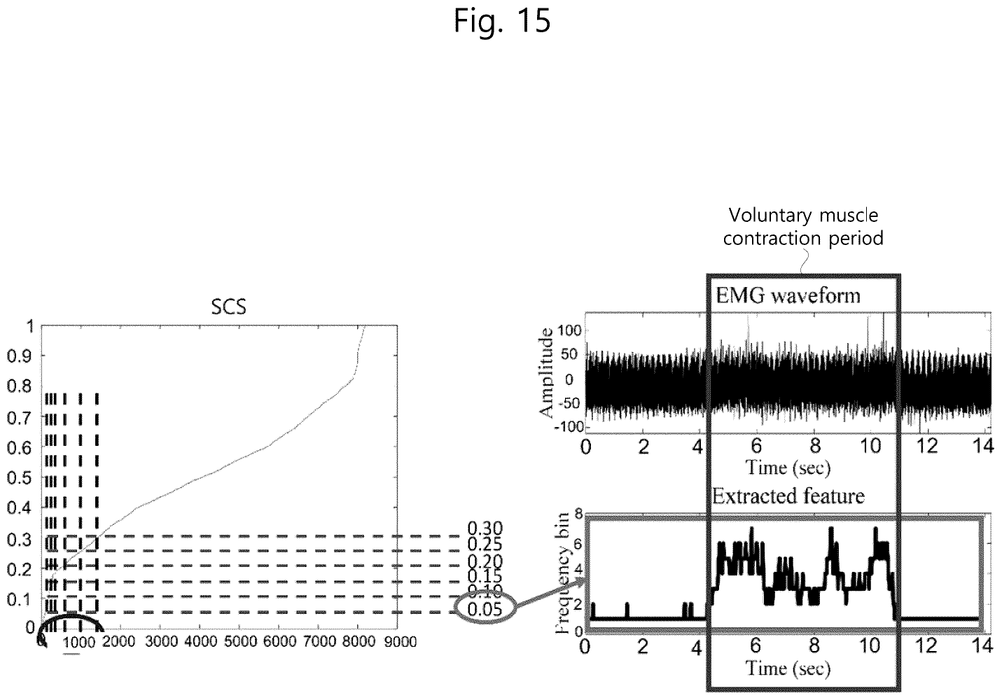

FIG. 15 is a graph showing a method of extracting a percentile of spectral cumulative sum PoSCS. Referring to FIG. 15, the extraction of the percentile of spectral cumulative sum PoSCS may be exemplarily performed through the following process.

First, after accumulating magnitude in the positive x-axis direction in the frequency domain, max-normalization data is used. Then, after shifting the horizontal line from 0.05 to 0.30 in units of 0.05 based on the y-axis, a frequency bin of the contact point between the horizontal line and the cumulative spectrum sum SCS is extracted as a feature. By extracting a 6-order feature vector for each frame, the use of the spectral cumulative sum SCS can be used to effectively distinguish between involuntary and voluntary muscle contractions. This process is shown in the graph on the right, which shows the extracted characteristics of frequency bins.

The noise component in the frequency domain caused by involuntary muscle contraction has an abnormally bouncing value, different from the frequency component of voluntary muscle contraction. Therefore, when involuntary and voluntary muscle contractions are present at the same time, the characteristics of the cumulative sum of spectra SCS are different from that when only involuntary muscle contractions are present. In addition, the noise component in the frequency domain generated due to involuntary muscle contraction appears differently depending on the frequency parameter of the electrical stimulation ES. Characteristics that appear prominently in the voluntary muscle contraction section are different according to the electrical stimulation environment. Therefore, for all electrical stimulation environments, in order to construct a high-performance model, as mentioned above, a multi-dimension type feature vector should be utilized. As a result, it can be confirmed that the percentile of spectral cumulative sum PoSCS is prominent in the voluntary muscle contraction section.

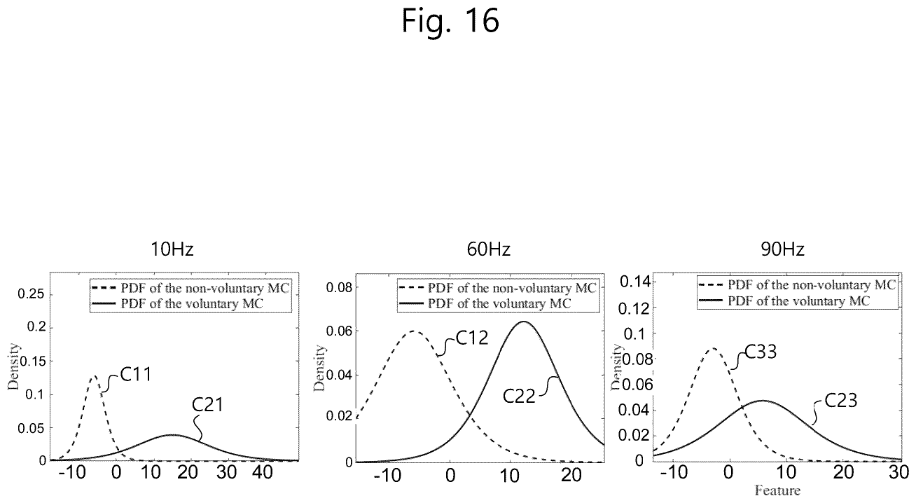

FIG. 16 is a probability density function PDF showing the results of extracting the percentile of spectral cumulative sum PoSCS for each frequency from the EMG signal (EMG). Referring to FIG. 16, in the EMG signal in the time domain, involuntary muscle contraction and voluntary muscle contraction may be distinguished in each frequency band. However, it can be seen that overlapping portions exist. Therefore, it can be seen that a separation operation using an artificial intelligence algorithm is necessary.

After extracting the percentile of spectral cumulative sum PoSCS for each frequency, the probability density function PDF of the characteristic for the involuntary muscle contraction signal is appeared to curves C11, C12, C13 at each frequency 10 Hz, 60 Hz, 90 Hz. And the probability density function of the characteristic for the voluntary muscle contraction signal is represented by curves C21, C22, and C23 at each frequency 10 Hz, 60 Hz, 90 Hz. According to the extraction result of the percentile of spectral cumulative sum PoSCS, the involuntary and voluntary muscle contraction signals at low frequencies have different averages, making it possible to distinguish them relatively clearly. However, since the extracted features overlap each other, deep learning or artificial intelligence techniques for the extracted features are needed to provide high classification resolution at any frequency. In particular, in the present invention, a Long Short Term Memory LSTM algorithm that provides the highest performance for long-term time series data will be used.

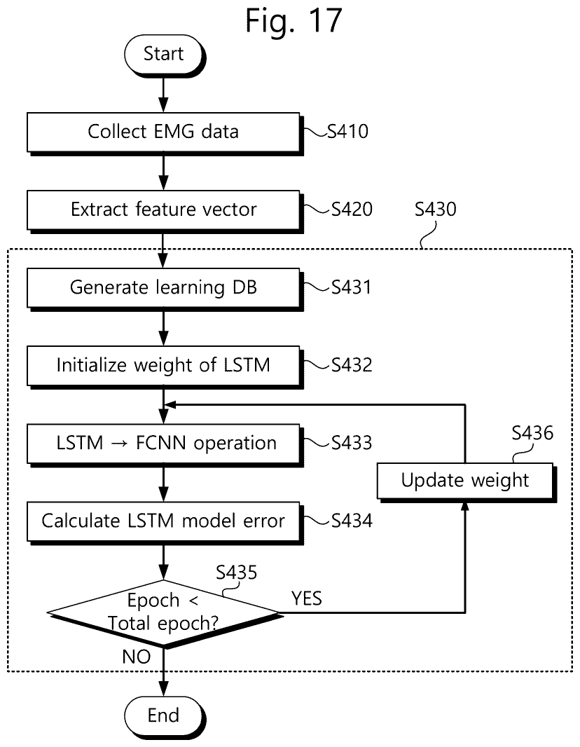

FIG. 17 is a flowchart illustrating a learning method of an artificial intelligence calculating unit for separating a voluntary muscle contraction signal and an involuntary muscle contraction signal according to an embodiment of the present invention. Referring to FIG. 17, the voluntary/involuntary muscle contraction detection unit 2110 (refer to FIG. 13) may learn LSTM, which is a type of a recurrent neural network (RNN), using an input EMG signal. Through learning, it is possible to discriminate at high resolution between voluntary and involuntary muscle contraction signals.

In step S410, the voluntary/involuntary muscle contraction detection unit 2110 may collect electromyography EMG data. The voluntary/involuntary muscle contraction detection unit 2110 may apply electrical stimulation ES to body muscles and measure electromyography EMG data.

In step S420, the voluntary/involuntary muscle contraction detecting unit 2110 may analyze EMG data and extract a feature vector. The voluntary/involuntary muscle contraction detector 2110 may remove a noise signal included in the electromyography EMG data, and then extract a feature vector related to muscle strength or muscular endurance.

In step S430, the voluntary/involuntary muscle contraction detection unit 2110 learns the artificial intelligence AI model based on the feature vector. The voluntary/involuntary muscle contraction detection unit 2110 generates learning data for artificial intelligence learning. The voluntary/involuntary muscle contraction detecting unit 2110 may generate a learning database DB based on the feature vector (S431). The voluntary/involuntary muscle contraction detecting unit 2110 may initialize the LSTM weight (S432). The voluntary/involuntary muscle contraction detection unit 2110 shuffles the learning database DB. That is, the voluntary/involuntary muscle contraction detection unit 2110 may provide training data to a fully connected neural network (FCNN) and process it as a learning operation (S433). The voluntary/involuntary muscle contraction detecting unit 2110 may calculate a current LSTM model error (S434). The voluntary/involuntary muscle contraction detection unit 2110 determines whether the error (Epoch) learned so far is smaller than the total error (Total epoch)(S435). The voluntary/involuntary muscle contraction detection unit 2110 ends if the epoch learned so far is not less than the total epoch (NO). On the other hand, the voluntary/involuntary muscle contraction detector 2110 updates the LSTM weight (S436) if the epoch learned so far is less than the total epoch (YES), and returns to step S433.

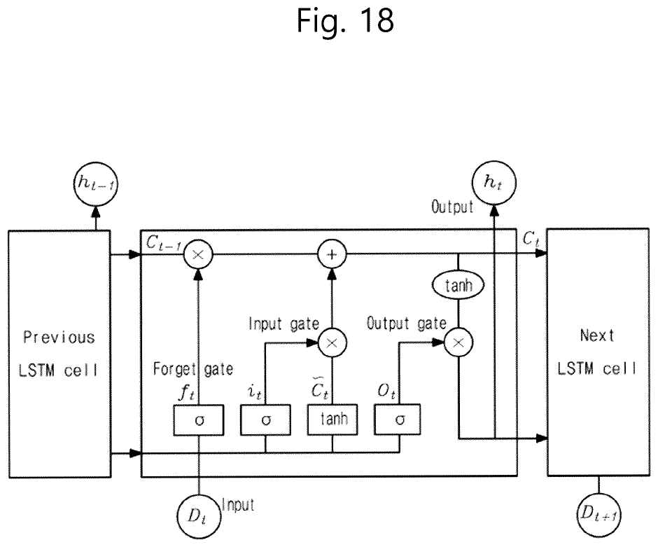

FIG. 18 is a diagram schematically showing the structure of an LSTM algorithm for discriminating a voluntary muscle contraction signal and an involuntary muscle contraction signal through sequential electromyography EMG data in the time domain of the present invention. Referring to FIG. 18, an update of weights, i.e., learning, of electromyography EMG data sequentially provided in time by the LSTM algorithm is performed.

The structure of the LSTM algorithm consists of LSTM cells that sequentially process input data Dt. Each of the LSTM cells determines how much of the past data to store or discard based on the current state, and reflects the current output to the result and delivers it to the next LSTM cell. For this function, one LSTM cell is composed of a forget gate, an input gate, and an output gate for processing the current input data Dt.

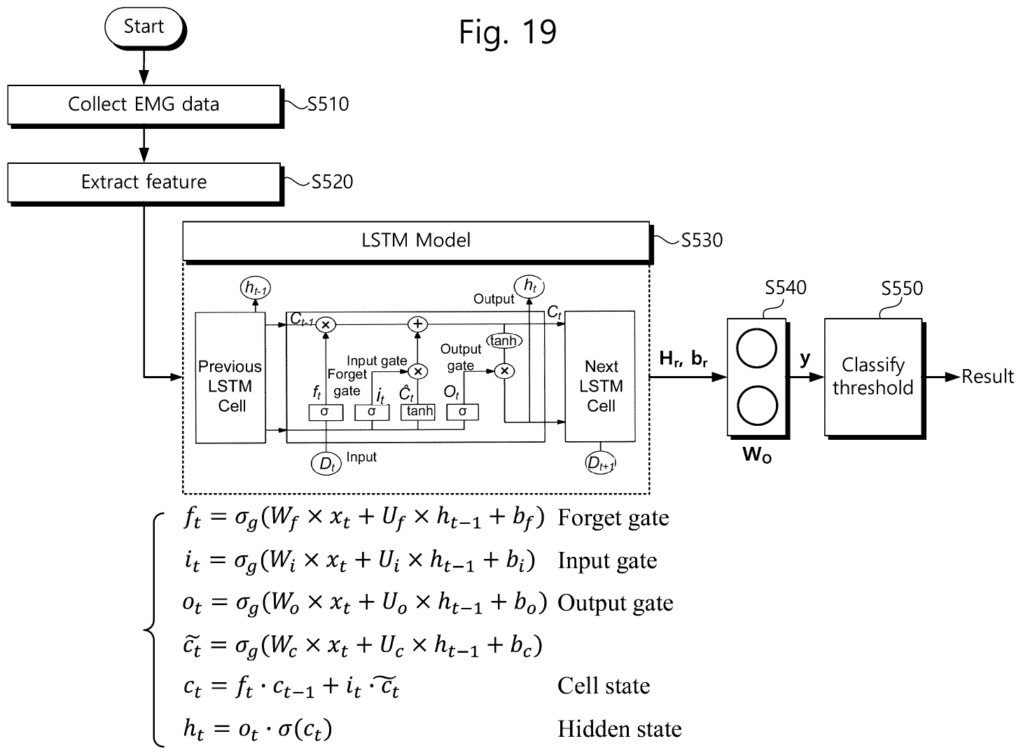

FIG. 19 is a flowchart showing an actual operation and a test operation of the voluntary/involuntary muscle contraction detecting unit of the present invention. Referring to FIG. 19, the voluntary/involuntary muscle contraction detection unit 2110 is configured to distinguish between a voluntary muscle contraction signal and an involuntary muscle contraction signal using the LSTM learned in FIG. 18 based on the input EMG signal.

In step S510, the voluntary/involuntary muscle contraction detection unit 2110 may collect electromyography EMG data. The voluntary/involuntary muscle contraction detection unit 2110 may apply electrical stimulation ES to body muscles and measure electromyography EMG data.

In operation S520, the voluntary/involuntary muscle contraction detection unit 2110 may analyze EMG data and extract a feature vector. The voluntary/involuntary muscle contraction detector 2110 may remove a noise electrical signal included in the electromyography EMG data, and then extract a feature vector related to muscle strength or muscular endurance.

In step S530, the voluntary/involuntary muscle contraction detection unit 2110 performs an LSTM operation based on the feature vectors that are sequentially input in time series. In step S540, the parameter Wo of the output layer provided as a result of the LSTM operation is provided. The voluntary/involuntary muscle contraction detection unit 2110 provides an output value ‘y’ using the parameter Wo. In step S550, the voluntary/involuntary muscle contraction detection unit 2110 finally performs classification using a threshold using the output value ‘y’, and outputs the result.

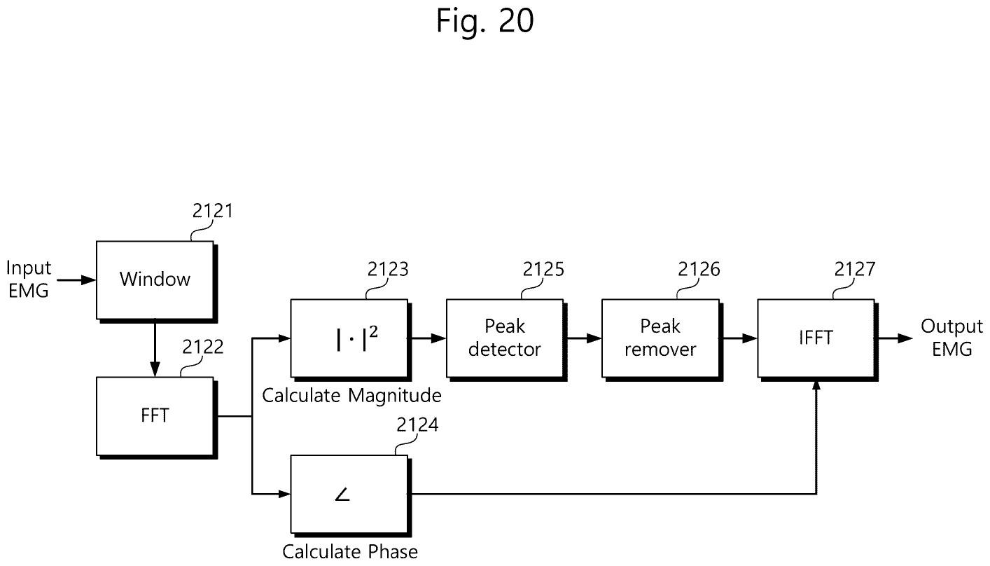

FIG. 20 is a block diagram exemplarily showing the involuntary muscle contraction signal removal unit shown in FIG. 13. Referring to FIG. 20, the involuntary muscle contraction signal removal unit 2120 includes a window unit 2121, a Fast Fourier Transform FFT 2122, and a magnitude calculator 2123 and phase calculator 2124, a peak detector 2125, a peak remover 2126, and an inverse FFT (IFFT) unit 2127.

The window unit 2121 performs windowing of an input signal (e.g., an EMG signal) of a time domain into a signal of a frequency domain. The window unit 2121 may shift in units of 20 samples in real time, configure a frame in units of 512 samples, and operate with an FFT size of 512. The fast Fourier transform unit 2122 performs the Fast Fourier Transform, and the calculators 2123 and 2124 calculate magnitude and phase. The peak detector 2125 and the peak remover 2126 detect noise by detecting the peak of the waveform, and perform peak suppression through substitution. The involuntary muscle contraction component appears as a peak-like magnitude like as an impulse. Accordingly, the peak detector 2125 detects an involuntary muscle contraction component having a magnitude like an impulse. The peak remover 2126 replaces the detected peak with an eps (=2.2204e-16) value, and then performs IFFT to obtain a signal from which the involuntary muscle contraction component is removed. The inverse FFT nit 2127 performs inverse transform using the magnitude of the waveform that has passed through the peak detector 2125 and the peak remover 2126 and the phase of the previously calculated waveform, and generates an output signal.

As a result, the involuntary muscle contraction signal removal unit 2120 uses an adaptive noise suppression algorithm that detects and then removes a peak signal related to the involuntary muscle contraction signal in the frequency domain. As the frequency of the electrical stimulation ES changes, the frequency component of the involuntary muscle contraction signal also changes. By using such an adaptive noise suppression algorithm, it is possible to effectively remove involuntary muscle contraction signals of varying frequencies. When a method in which the filter band is fixed is used, the involuntary muscle contraction signal removal unit 2120 using the adaptive noise suppression algorithm can provide stable performance because performance deviation may occur depending on the situation or the user.

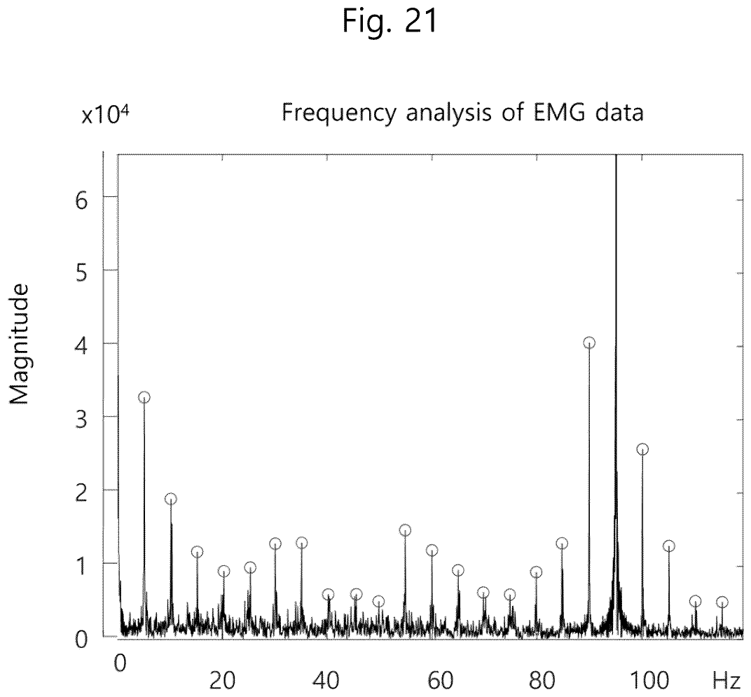

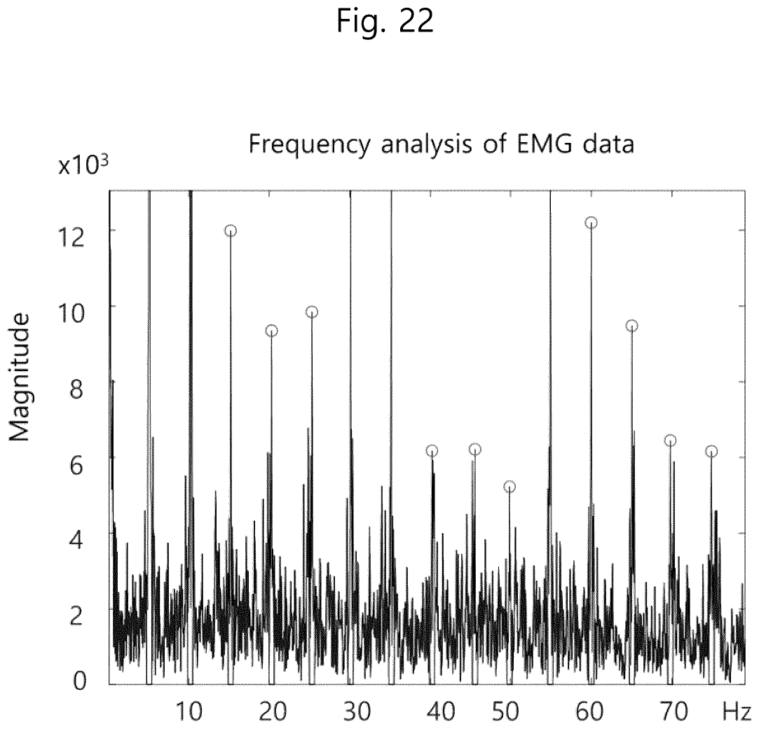

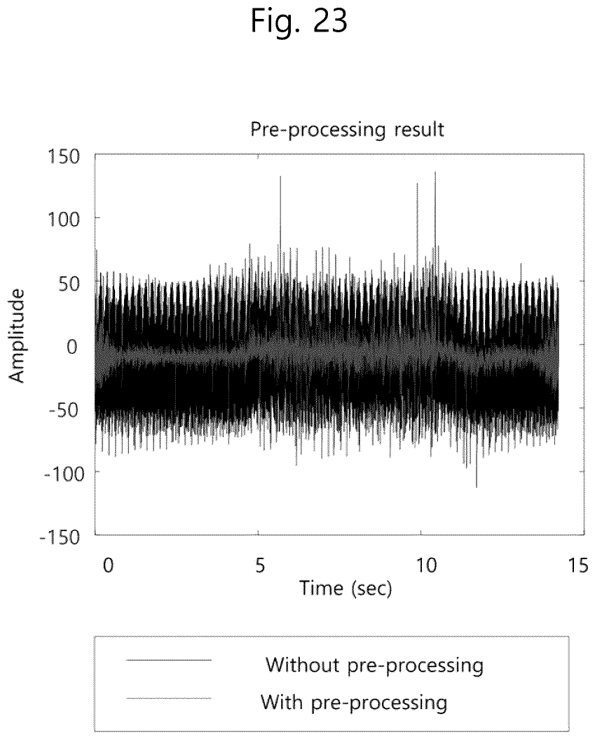

FIG. 21 and FIG. 22 are graphs showing results of frequency analysis of EMG data in the peak detector 2125 and the peak suppressor 1126. FIG. 23 is a graph showing a waveform (dotted line) with a pre-processing process in the inverse transform unit 2127 and a waveform without a pre-processing process (black).

The involuntary muscle contraction signal removal unit 2120 may be implemented as an adaptive noise suppression algorithm in the form of finding and removing a peak signal related to involuntary muscle contraction (i.e., noise) in the frequency domain. As the frequency of electrical stimulation changes, the frequency component of involuntary muscle contraction changes. In order to effectively remove the involuntary muscle contraction signal removal unit 2120, the involuntary muscle contraction component may be adaptively removed.

If a fixed filter is used, performance deviation may occur depending on the situation or person. The involuntary muscle contraction signal removal unit 2120 is to solve this problem, and it is possible to reduce the performance deviation depending on the situation or person.

The involuntary muscle contraction signal removal unit 2120 may operate in the following manner. For example, the involuntary muscle contraction signal removal unit 2120 shifts in units of 20 samples in real time, constitutes a frame in units of 512 samples, and drives the algorithm by setting the FFT size to 512. The involuntary muscle contraction signal removal unit 2120 performs FFT on a predefined frame, calculates a magnitude and a phase, and removes an involuntary muscle contraction component that appears like an impulse in magnitude.

The involuntary muscle contraction signal removal unit 2120 replaces the peak with an eps (=2.2204e-16) value in order to remove the involuntary muscle contraction component, and then performs IFFT to remove the involuntary muscle contraction component.

The involuntary muscle contraction signal removing unit 2120 may obtain a signal by removing the magnitude by 6 dB when it is classified as involuntary muscle contraction and is not a contraction period.

Referring to FIG. 23, the electrical stimulation treatment system 2100 using the electromyography-based functional electrical stimulation ECF of the present invention may provide a high efficiency of removing involuntary muscle contraction signals. The involuntary muscle contraction signal included in the EMG signal in the time domain can be effectively removed by applying an adaptive noise suppression algorithm. Accordingly, the electrical stimulation treatment system 1100 (refer to FIG. 12) of the present invention can generate functional electrical stimulation ECF based on low-noise voluntary muscle contraction signals. Therefore, highly reliable functional electrical stimulation treatment is possible without relying on specialists or expensive devices.

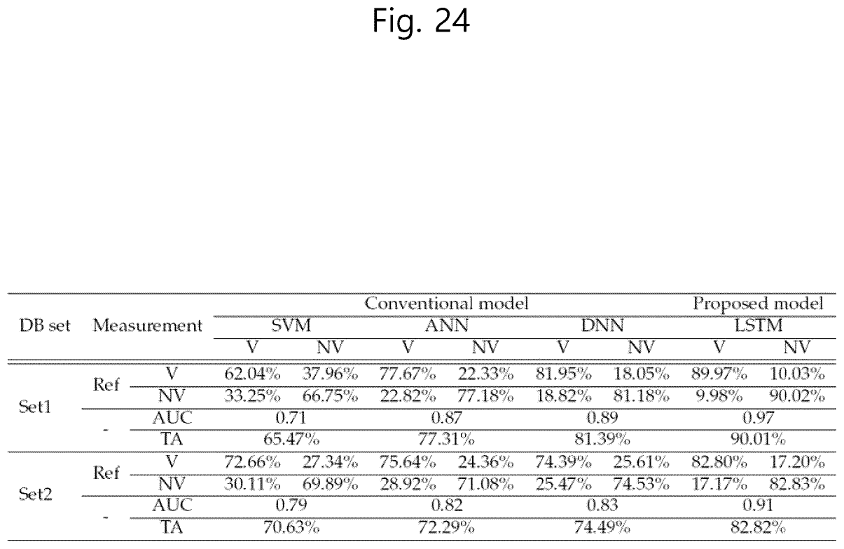

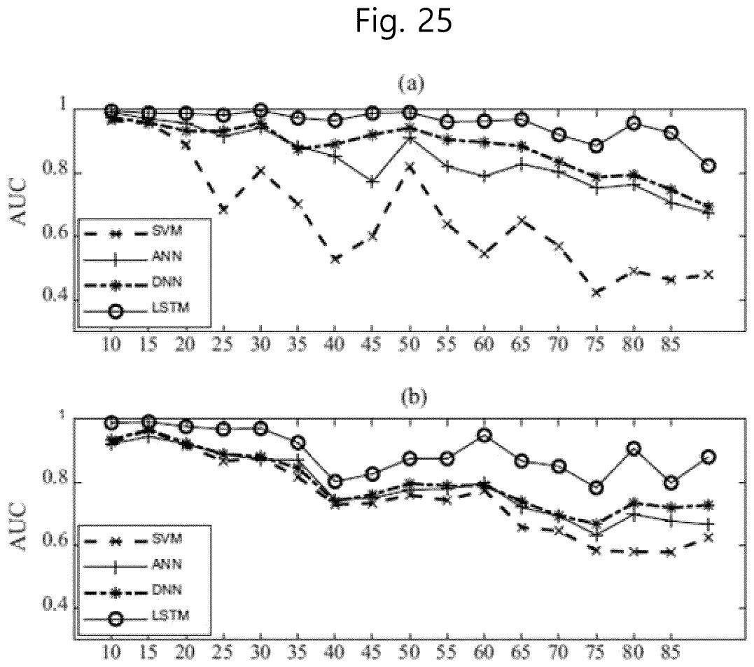

FIG. 24 and FIG. 25 are diagrams showing the results of testing the performance of the electrical stimulation treatment system for generating functional electrical stimulation FES based on the muscle stimulation signal of the present invention. For the test of the electrical stimulation treatment system 2100 of the present invention, electrical stimulation ES was applied while increasing the frequency from Hz to 90 Hz in 5 Hz increments. And while the electromyography signal EMG was collected while the electrical stimulation ES was provided, the measurement subject rested for 10 seconds, voluntary muscle contraction was performed for 20 seconds, and the pattern of resting for 10 seconds was repeated. A total of about 40 seconds of data are collected per person, and a database was built using the data collected in the same way for a total of 6 people. And the section in which voluntary muscle contraction was maintained was leveled to ‘l’, and the section in which only involuntary muscle contraction was present was leveled to ‘0’.

The artificial intelligence model uses all the extracted features as inputs, and, in addition, the initialization of the artificial intelligence model uses a random initialization method, and fine-tuning uses the error backpropagation method, and the number of fully connected layers is set to 1, and the number of units is set to 1. In addition, an adaptive moment estimation (Adam: Adaptive Momentum Estimation) method was used as an optimization algorithm for determining a weight update method. In addition, as the cost function, binary cross entropy, and as the active function, hyperbolic tangent, the number of cells is 3, and each cell has 128, 64, 32 hidden units were used.

Referring to FIG. 24, the performance of removing the involuntary muscle contraction signal in the case of using the LSTM, which is the artificial intelligence model of the present invention, and the case of applying the general artificial intelligence models (SVM, ANN, DNN) is shown in a table. According to the test results for the two groups (Set1, Set2), the total accuracy (TA) was the best at 90.01% and 82.82%, respectively, in the case of using the LSTM model.

Referring to FIG. 25, the results of the evaluation of the performance by frequency (AUC: Area under the Curve) of the artificial intelligence models for the two groups (Set1, Set2) are shown in a graph. In the case of using the LSTM model for each of the two groups (Set1, Set2), the highest reliability (AUV) was observed across all experimental frequencies.

The above descriptions are specific embodiments for carrying out the present invention. In addition to the above-described embodiments, the present invention will also include simple design changes or easily changeable embodiments. In addition, the present invention will include techniques that can be easily modified and implemented using the embodiments. Therefore, the scope of the present invention should not be limited to the above-described embodiments, but should be defined by the claims described below as well as the claims and equivalents of the present invention.

What is claimed is:

1. A sarcopenia diagnostic system, comprising:
an electrical stimulation and measurement unit configured to apply multi-frequency electrical stimulation to a body and measure a multi-frequency impulse response signal m-FIRS to the multi-frequency electrical stimulation;
a response signal analysis unit configured to remove noise and distortion from the multi-frequency impulse response signal m-FIRS to obtain an involuntary muscle contraction signal, and configured to extract a feature vector in each of time domain and frequency domain from the involuntary muscle contraction signal; and
an artificial intelligence model learning unit receiving the extracted feature vector as input, and generates a classification for muscle strength and muscular endurance from the feature vector through artificial intelligence-based model learning to diagnose sarcopenia,
wherein the multi-frequency impact response signal m-FIRS is provided in units of a plurality of segments divided by frequency.

2. The system of claim 1, wherein the response signal analysis unit includes:
an electrical stimulation filter for extracting the involuntary muscle contraction signal by performing a pre-processing operation to remove a noise signal or a distortion included in the multi-frequency impact response signal m-FIRS; and
a feature extraction unit for extracting the feature vector related to muscle strength or muscular endurance based on the involuntary muscle contraction signal provided from the electrical stimulation filter.

3. The system of claim 1, wherein the feature vector in the time domain includes at least one of a feature used in a specific muscle diagnostic equipment, an envelope characteristics, a waveform pattern and shape, and a level crossing rate, and
wherein the feature vector in the frequency domain includes at least one of a Percentile of Spectral Cumulative Sum (PoSCS), a Log Power Spectrum, a Percentile Pattern of Spectral Cumulative Sum (PPoSCS), and a log power spectrum shift.

4. The system of claim 3, wherein the feature used in a specific muscle diagnostic equipment includes at least one of a muscle tone state, a stiffness of a muscle, a decrement indicating the elasticity of the muscle, a relaxation time of the muscle, and a creep of the muscle.

5. The system of claim 1, wherein the artificial intelligence model learning unit includes a deep learning model using at least one of an initialization method of a random initialization method, a fine tuning of a backpropagation method, and an optimization algorithm of an adaptive moment estimation Adam, a cost function of Minimum Mean Square Error MMSE, and an active function of an exponential linear unit ELU.

6. An electrical stimulation treatment system for controlling and generating a functional electrical stimulation signal by collecting an electromyography signal generated in response to electrical stimulation from a body, the system comprising:
a voluntary/involuntary muscle contraction detection unit that extracts a feature vector from the frequency domain of the electromyography signal and distinguishes and detects a voluntary muscle contraction signal and an involuntary muscle contraction signal from the extracted feature vector by applying an artificial intelligence model;
an involuntary muscle contraction signal removal unit configured to remove the involuntary muscle contraction signal from the electromyography signal according to the detection result;
a muscle activity intensity calculator configured to calculate a root mean square RMS of the electromyography signal from which the involuntary muscle contraction signal is removed; and
a functional electrical stimulation control unit that compares the effective value with a threshold value and generates the functional electrical stimulation signal to be applied to the body according to the comparison result.

7. The system of claim 6, wherein the feature vector includes at least one of a percentile of spectral cumulative sum PoSCS and a log power spectrum detected in the frequency domain of the electromyography signal.

8. The system of claim 6, wherein the involuntary muscle contraction signal removal unit attenuates the section including the involuntary muscle contraction signal of the electromyography signal by 6 dB to remove the involuntary muscle contraction signal.

9. The system of claim 6, wherein the artificial intelligence model distinguishes the involuntary muscle contraction signal and the voluntary muscle contraction signal from the electromyography signal by using an artificial intelligence algorithm.

10. The system of claim 6, wherein the involuntary muscle contraction signal removal unit comprises:
a window unit for selecting a window of the electromyography signal;
a fast Fourier transform unit for processing a signal included in the selected window by fast Fourier transform;
a magnitude and phase calculator for calculating magnitudes and phases of signals output from the fast Fourier transform unit, respectively;
a peak detector for detecting a peak in the magnitude of the signal; and
a peak removing unit for filtering a noise signal corresponding to the detected peak.
